(19) United States
(12) Patent Application Publication
(10) Pub. No.: US 2025/0305068 A1
(43) Pub. Date: Oct. 2, 2025

(54) NEW BIOMARKER FOR DISORDERS AND DISEASES ASSOCIATED WITH INTESTINAL DYSBIOSIS

(71) Applicant: PHARMABIOME AG, SCHLIEREN (CH)

(72) Inventors: TOMAS DE WOUTERS, ZÜRICH (CH); LAURA ANTHAMATTEN, BRIG-GLIS (CH); PHILIPP ROGALLA VON BIEBERSTEIN, ZÜRICH (CH); GABRIEL LEVENTHAL, ZÜRICH (CH)

(21) Appl. No.: 18/722,618

(22) PCT Filed: Dec. 22, 2022
(86) PCT No.: PCT/EP2022/087530

§ 371 Date: Jun. 21, 2024

(30) Foreign Application Priority Data
Dec. 22, 2021 (EP) 21217015.3

(51) Int. Cl. C12Q 1/689; C12Q 1/6874

(52) U.S. Cl. CPC C12Q 1/689; C12Q 1/6874; C12Q 2600/112

(57) ABSTRACT

The present invention relates to a new biomarker of inflammatory bowel disease and its uses. The new biomarker is a ratio of Dialister spp. to Phascolarctobacterium spp. This biomarker can be used for diagnosis purpose of diseases or disorders associated with an intestinal dysbiosis, for stratifying patient population in a clinical trial, for selecting a subject that will benefit from a treatment, or for monitoring treatment response. The present invention further provides new therapeutic strategies.

### CROSS-REFERENCE TO RELATED APPLICATION

This application is the U.S. national stage application of International Patent Application No. PCT/EP2022/087530, filed Dec. 22, 2022.

### REFERENCE TO SEQUENCE LISTING

The Sequence Listing for this application is labeled “Seq-List-replace.xml” which was created on Aug. 21, 2024 and is 38,845 bytes. The entire content of the sequence listing is incorporated herein by reference in its entirety.

### FIELD OF THE INVENTION

The present invention relates to the field of microbiology and diagnostics. It provides important contributing biomarkers for diseases associated with intestinal dysbiosis, in particular for inflammatory bowel disease (IBD).

### BACKGROUND OF THE INVENTION

The gut microbiota is composed of more than a thousand different bacterial species. It is known to play a beneficial role for the host by exerting many biological functions, such as nutrient absorption, maintenance of intestinal epithelium integrity, protection from pathogens or homeostasis of immune responses. Studies have shown that a persistent or transient imbalance of the gut's microbial community, commonly referred to as dysbiosis, relates to several diseases, such as inflammatory bowel disease (IBD) or irritable bowel syndrome (IBS).

One of the key roles of the intestinal microbiota is the fermentation of complex carbohydrates into short-chain fatty acids (SCFA) that then become available to the host. SCFA are organic fatty acids with one to six carbons that are produced within the intestinal lumen by bacterial fermentation of undigested dietary carbohydrates. Acetate, propionate, and butyrate are the most abundant SCFA produced in the gastrointestinal tract (GIT).

For bacterial fermentation of undigested dietary carbohydrates, the individual metabolic steps of the fermentation cascade are often distributed across different bacteria, whereby intermediate metabolites are produced by one bacterium and cross-fed to another. While the end products of this fermentation—especially butyrate and propionate—have been associated with good health, the accumulation of intermediate metabolite products—especially formate, lactate, and succinate—have been associated with poor health and microbiome-associated disease.

In particular, butyrate is known to have beneficial effects on epithelial barrier function and overall gut health. It is a cellular mediator regulating multiple functions of gut human and microbial cells including gene expression, immune modulation and oxidative stress reduction.

Increased levels of the intermediate metabolite succinate within the gut lumen, however, have been hypothesized to contribute to intestinal inflammation (Macias-Ceja et al., Mucosal Immunol. 2019 Jan. 12 (1): 178-187; Fernández-Veledo and Vendrell, Rev Endocr Metab Disord, 2019 Dec. 20 (4): 439-447; Zakerska-Banaszak et al., Sci Rep 11, 2021, 2166).

Only a few bacterial taxa are known to be able to metabolize succinate in the intestine. These bacteria are conserved to two phylogenetic clades: (i) various strains within the Bacteroidetes phylum (e.g., Bacteroides spp., Parabacteroides spp., Prevotella spp., Alistipes spp.), and (ii) various strains within the Veillonellaceae family (e.g., Dialister spp., Phascolarctobacterium spp.). However, while the Bacteroides typically encode the genes to metabolize intracellular succinate into propionate, they are not typically able to grow on succinate as an external carbon source. In contrast, members of the Veillonellaceae family are able to grow on extracellular succinate. Thus, the genera Dialister and Phascolarctobacterium are considered to be the most common succinate-utilizers within the human microbiome.

The relative abundance of these succinate-utilizing bacteria has been previously associated with different disease indications. Decreased relative abundance of Phascolarctobacterium was measured in patients suffering from intestinal bowel disease compared to healthy individuals (Morgan et al., Genome Biol. 2012; 13 (9): R79).

Increased relative abundance of Dialister was correlated with an increased disease activity score in Spondyloarthritis patients (Tito et al., Arthritis Rheumatol 2016, 69 (1)). How these compositional signatures mechanistically translate to microbiome succinate metabolism is yet unknown.

WO2019/141780 discloses kits and methods for determining the ratio of succinate-producing bacteria to succinate-consuming bacteria in the intestinal tract of a patient. Elevated levels of circulating succinate in a subject have been detected in several high-risk cardiovascular diseases. Succinate produced by bacteria of the gut microbiota is an essential contributor to total circulating succinate levels. The ratio of succinate-producing bacteria to succinate-consuming bacteria, in particular the (Prevotellaceae+Veillonellaceae)/(Odoribacteriaceae+Clostridiaceae) ratio, measured in a stool sample from a subject can be related to circulating succinate levels in the same subject. The kit comprises primer sets to detect at least one succinate-producing bacterium and at least one succinate-consuming bacterium.

WO2017/109059 discloses methods for detecting the presence or assessing the risk of development of inflammatory arthritis diseases based on gut microbial markers. When the gut microbiome profile of said patient indicates the presence of Dialister spp., this results in a conclusion that said patient has or is at risk of developing inflammatory arthritis disease, or joint inflammation or gut inflammation or more particular spondyloarthritis.

There is a constant need of biomarkers for diseases associated with intestinal dysbiosis such as inflammatory bowel disease (IBD).

### SUMMARY OF THE INVENTION

The inventors have surprisingly found that the identity of the dominant succinate-utilizing bacterium in an individual is an important contributing biomarker for diseases or disorders associated with intestinal dysbiosis, preferably such as IBD. More particularly, a ratio of Dialister spp. to Phascolarctobacterium spp. (or Phascolarctobacterium spp. to Dialister spp.) is a biomarker for IBD, colorectal cancer (CRC), atherosclerotic cardiovascular disease (ACVD) and underweight (low body mass index; BMI).

The inventors observed that the ratio of Dialister spp. to Phascolarctobacterium spp. (or Phascolarctobacterium spp. to Dialister spp.) is able to significantly differentiate between microbiomes of diseased subjects suffering of diseases or disorders associated with intestinal dysbiosis, preferably such as IBD and microbiomes of healthy subjects, while in contrast simply quantifying the abundance of succinate-utilizing bacteria was not able to differentiate between them.

Accordingly, the present invention relates to the use of a ratio of Dialister spp. to Phascolarctobacterium spp. (D/P) or a ratio of Phascolarctobacterium spp. to Dialister spp. (P/D) as a biomarker, in particular for diagnosis purpose of a disease or disorder associated with an intestinal dysbiosis, preferably IBD, for stratifying patient population in a clinical trial, or for monitoring treatment response. Preferably, the present invention relates to the use of a ratio of Dialister spp. to Phascolarctobacterium spp. (D/P) or a ratio of Phascolarctobacterium spp. to Dialister spp. (P/D) as a biomarker for diagnosis purpose of a disease or disorder associated with an intestinal dysbiosis, for stratifying patient population suffering from a disease or disorder associated with an intestinal dysbiosis in a clinical trial, or for monitoring treatment response for a patient suffering from a disease or disorder associated with an intestinal dysbiosis.

In some embodiments, the biomarker is a composite biomarker comprising:
the ratio of Dialister spp. to Phascolarctobacterium spp. (D/P) or the ratio of Phascolarctobacterium spp. to Dialister spp. (P/D); and
the presence or absence of Prevotella spp.

The invention also concerns method for assessing whether a subject is suffering or is at risk of developing a disease or disorder associated with an intestinal dysbiosis, comprising:
a) providing a biological sample from the subject;
b) determining a ratio of Dialister spp. to Phascolarctobacterium spp. (D/P) or a ratio of Phascolarctobacterium spp. to Dialister spp. (P/D) in the biological sample; and
c) classifying the subject as suffering or being at risk of developing the disease or disorder associated with an intestinal dysbiosis based on the determined ratio of Dialister spp. to Phascolarctobacterium spp. or the determined ratio of Phascolarctobacterium spp. to Dialister spp., in particular if the ratio D/P is superior to 1 or the ratio P/D is inferior to 1.

Preferably, the disease or disorder associated with an intestinal dysbiosis is inflammatory bowel disease (IBD), and the subject is classified as suffering or being at risk of developing inflammatory bowel disease (IBD) if the ratio D/P is superior to 1 or the ratio P/D is inferior to 1.

Optionally, the method further comprises the determination of the presence or absence of Prevotella spp. in the biological sample.

Preferably, the disease or disorder associated with an intestinal dysbiosis is inflammatory bowel disease (IBD), and the method further comprises the determination of the presence or absence of Prevotella spp. in the biological sample, and the subject is classified as suffering or being at risk of developing inflammatory bowel disease (IBD) if the ratio D/P is superior to 1 or the ratio P/D is inferior to 1, and Prevotella is absent.

In a particular aspect, the present invention relates to a method for assessing whether a subject is suffering or is at risk of developing inflammatory bowel disease (IBD), comprising
a) providing a stool sample from the subject;
b) determining a ratio of Dialister spp. to Phascolarctobacterium spp. in the stool sample; and
c) classifying the subject as suffering or being at risk of developing inflammatory bowel disease (IBD) based on the determined ratio of Dialister spp. to Phascolarctobacterium spp., in particular if the ratio is higher than 1.

Optionally, the subject is classified as suffering or being at risk of developing inflammatory bowel disease (IBD) if the ratio is higher than 1.05, 1.1, 1.2 or 1.3.

The invention also concerns a method for monitoring treatment response in a subject suffering of a disease or disorder associated with an intestinal dysbiosis, comprising:
a) providing a first biological sample from the subject at a first time;
b) determining a first ratio of Dialister spp. To Phascolarctobacterium spp. in the first biological sample;
c) providing a second biological sample from the subject at a second time, the second time being after the first time;
d) determining a second ratio of Dialister spp. To Phascolarctobacterium spp. in the second biological sample; and
e) comparing the first ratio and the second ratio, preferably wherein a decrease of the ratio being indicative of an effective treatment of said disease or disorder associated with an intestinal dysbiosis in the subject.

In particular, the disease or disorder associated with an intestinal dysbiosis is inflammatory bowel disease (IBD), and a decrease of the D/P ratio is indicative of an effective treatment of IBD in the subject.

Alternatively, the invention relates to a method for monitoring treatment response in a subject suffering of a disease or disorder associated with an intestinal dysbiosis, comprising:
a) providing a first biological sample from the subject at a first time;
b) determining a first ratio of Phascolarctobacterium spp. to Dialister spp. in the first biological sample;
c) providing a second biological sample from the subject at a second time, the second time being after the first time;
d) determining a second ratio of Phascolarctobacterium spp. to Dialister spp. in the second biological sample; and
e) comparing the first ratio and the second ratio, preferably an increase of the ratio being indicative of an effective treatment of said disease or disorder associated with an intestinal dysbiosis in the subject.

In particular the disease or disorder associated with an intestinal dysbiosis is inflammatory bowel disease (IBD), and an increase of the P/D ratio is indicative of an effective treatment of IBD in the subject.

In an additional aspect, the present invention relates to a method for monitoring treatment response in a subject suffering of IBD, comprising:
a) providing a first stool sample from the subject at a first time, for instance before the beginning of a treatment or during the treatment;
b) determining a first ratio of Dialister spp. to Phascolarctobacterium spp. in the first stool sample;
c) providing a second stool sample from the subject at a second time, for instance during the treatment or at the end of the treatment, the second time being after said beginning and said first time;
d) determining a second ratio of Dialister spp. to Phascolarctobacterium spp. in the second stool sample; and
e) comparing the first ratio and the second ratio, a decrease of the ratio being indicative of an effective treatment of IBD in the subject.

The invention also concerns a method for selecting a subject suffering from a disease or disorder associated with an intestinal dysbiosis as susceptible to benefit from a treatment comprising the administration of Phascolarctobacterium spp., wherein the method comprises:
a) providing a biological sample from the subject;
b) determining a ratio of Dialister spp. to Phascolarctobacterium spp. (D/P) or a ratio of Phascolarctobacterium spp. to Dialister spp. (P/D) in the biological sample; and,
c) selecting the subject based on the determined ratio of Dialister spp. to Phascolarctobacterium spp. (D/P) or Phascolarctobacterium spp. to Dialister spp. (P/D) as susceptible to benefit from the treatment, in particular if the D/P ratio is superior to 1 or the P/D ratio is inferior to 1.

Preferably, the disease or disorder associated with an intestinal dysbiosis is inflammatory bowel disease (IBD), and the subject is susceptible to benefit from the treatment if the ratio D/P is superior to 1 or the P/D ratio is inferior to 1.

Optionally, the method further comprises the determination of the presence or absence of Prevotella spp. in the biological sample.

Particularly, when the disease or disorder associated with intestinal dysbiosis is IBD such method further comprises the determination of the presence or absence of Prevotella spp. in the biological sample, and the subject is susceptible to benefit from the treatment if i) the ratio D/P is superior to 1 or the P/D ratio is inferior to 1, and ii) Prevotella is absent.

The present invention also relates to a method for selecting a subject susceptible to benefit from a treatment including administering Phascolarctobacterium spp., comprising:
a) providing a stool sample from the subject;
b) determining a ratio of Dialister spp. to Phascolarctobacterium spp. in the stool sample; and
c) selecting the subject based on the determined ratio of Dialister spp. to Phascolarctobacterium spp., as susceptible to benefit from the treatment, in particular if the D/P ratio is higher than 1.

Preferably, the treatment is a dietary intervention, a treatment with a drug, or preferably a treatment with a probiotic administration, in particular a probiotic including Phascolarctobacterium spp., preferably Phascolarctobacterium faecium and/or Phascolarctobacterium_A succinatutens.

Even more preferably, the treatment comprises a probiotic including Phascolarctobacterium spp. and Prevotella spp. In particular the treatment comprises a probiotic including Phascolarctobacterium faecium and/or Phascolarctobacterium_A succinatutens and Prevotella spp.

The ratio of Dialister spp. to Phascolarctobacterium spp. may be the result of dividing total number, percentage, abundance or concentration of Dialister spp. by total number, percentage, abundance or concentration of Phascolarctobacterium spp., respectively.

The ratio of Phascolarctobacterium spp. to Dialister spp. may be the result of dividing total number, percentage, abundance or concentration of Phascolarctobacterium spp. by total number, percentage, abundance or concentration of Dialister spp., respectively.

Optionally, the abundance is measured using optical density, qPCR, flow cytometry, chamber counting, total bacterial DNA quantification or metagenomic sequencing and grouping of genes.

Preferably, the abundance is measured using optical density, qPCR, microarray technique, amplicon sequencing, single cell sequencing or single cell Matrix Assisted Laser Desorption Ionization-Time of Flight (MALDI-TOF) following flow cytometry or other microfluidic methods, chamber counting, total bacterial DNA quantification or metagenomic sequencing and grouping of genes, proteomic profiling or total RNA sequencing of complex samples. Even more preferably the abundance is measured by amplifying the V3/V4 region of the hypervariable regions of the 16S rRNA gene and by sequencing amplicons.

The invention also concerns a veterinary or pharmaceutical composition comprising Phascolarctobacterium spp. for use in the treatment of a subject having a disease or disorder associated with an intestinal dysbiosis, said subject having a Dialister spp. to Phascolarctobacterium spp. (D/P) ratio superior to 1 or a Phascolarctobacterium spp. to Dialister spp. (P/D) ratio inferior to 1. Such composition preferably comprises Phascolarctobacterium spp. and optionally Prevotella spp. Such veterinary or pharmaceutical composition can be used in combination with an agent able to decrease Dialister spp. population such as an antibiotic agent, selective nutrition, an agent to modulate transit time and/or for cleansing an in situ microbiome space.

The present invention further relates to a veterinary or pharmaceutical composition comprising Phascolarctobacterium spp. for use in the treatment of a subject having a Dialister spp. to Phascolarctobacterium spp. ratio superior to 1. Optionally, the veterinary or pharmaceutical composition is used in combination with an agent able to decrease Dialister spp. population such as an antibiotic agent.

The present invention further relates to a kit comprising detecting means designed to specifically detect Dialister spp. and Phascolarctobacterium spp. The detection means can be primer sets or probes specific for Dialister spp. and Phascolarctobacterium spp. The detection means can be primer sets or probes designed to specifically hybridize the 16S rRNA gene of Dialister spp. and Phascolarctobacterium spp., especially the hypervariable regions of the 16S rRNA gene of Dialister spp. and Phascolarctobacterium spp. The primers can also be designed to bind in the V3/V4 region and the amplified region is then classified as Dialister spp. or Phascolarctobacterium spp. by sequencing. The detection means could also be based on other specific marker genes derived from the genomes of Dialister spp. and Phascolarctobacterium spp. The detection means could also be characteristic protein profiles of Dialister spp. and Phascolarctobacterium spp. for use, for example, in MALDI-TOF. These alternative detection means are typically seen as interchangeable for taxonomic identification by persons skilled in the art. The present invention also relates to the use of the kit for detecting a ratio of Dialister spp. to Phascolarctobacterium spp. in a stool sample from a subject, for diagnosis purpose of inflammatory bowel disease (IBD), for stratifying patient population in a clinical trial, for selecting a subject that will benefit from a treatment, or for monitoring treatment response.

The invention particularly relates to a kit comprising detecting means designed to specifically detect i) Dialister spp. and Phascolarctobacterium spp., ii) optionally Prevotella spp., the means being preferably primer sets or probe(s) specific for detecting Dialister spp. and primer sets or probe(s) specific for detecting Phascolarctobacterium spp., and optionally primer sets or probe(s) specific for detecting Prevotella spp. Preferably, the kit comprises primer sets or probes designed to specifically hybridize the hypervariable regions of the 16S rRNA gene of Dialister spp. and Phascolarctobacterium spp., respectively, and optionally primer sets or probes designed to specifically hybridize the hypervariable regions of the 16S rRNA gene of Prevotella spp. The invention also concerns the use of such kit for detecting a ratio of Dialister spp. to Phascolarctobacterium spp. or a ratio of Phascolarctobacterium spp. to Dialister spp. and optionally the presence or absence of Prevotella spp. in a biological sample from a subject, preferably for diagnosis purpose of a disease or disorder associated with intestinal dysbiosis, for stratifying patient population suffering from a disease or disorder associated with an intestinal dysbiosis in a clinical trial, for selecting a subject suffering from a disease or disorder associated with an intestinal dysbiosis that will benefit from a treatment, or for monitoring treatment response for a patient suffering from a disease or disorder associated with an intestinal dysbiosis.

The present invention further relates to a kit of parts comprising i) an agent able to decrease Dialister spp. population in a subject and ii) an agent able to increase Phascolarctobacterium spp. population in a subject as a combined preparation for simultaneous, separate or sequential use, for use in the treatment of a subject having a Dialister spp. to Phascolarctobacterium spp. (D/P) ratio superior to 1 and/or suffering from IBD.

The invention also concerns a kit of parts comprising i) an agent able to decrease Dialister spp. population in a subject and ii) an agent able to increase Phascolarctobacterium spp. population in a subject as a combined preparation for simultaneous, separate or sequential use, for use in the treatment of a subject having an intestinal dysbiosis caused by a Dialister spp. to Phascolarctobacterium spp. (D/P) ratio superior to 1 or a Phascolarctobacterium spp. to Dialister spp. ratio inferior to 1.

The invention also relates to a method of treatment of a disease or disorder associated with an intestinal dysbiosis in a subject comprising administering a therapeutically effective amount of a veterinary or pharmaceutical composition comprising an agent capable of decreasing the ratio of Dialister spp. to Phascolarctobacterium spp. (D/P), or increasing the ratio of Phascolarctobacterium spp. to Dialister spp. (P/D) to said subject. The invention also concerns a veterinary or pharmaceutical composition comprising an agent capable of decreasing the ratio of Dialister spp. to Phascolarctobacterium spp. (D/P), or increasing the ratio of Phascolarctobacterium spp. to Dialister spp. (P/D) for the manufacture of a medicament for the treatment of a disease or disorder associated with an intestinal dysbiosis.

In particular the agent is i) an agent able to decrease Dialister spp. population, such as an antibiotic agent, an agent to modulate transit time and/or for cleansing an in situ microbiome space or ii) an agent able to increase Phascolarctobacterium spp. population, such as a probiotic comprising Phascolarctobacterium spp. and optionally Prevotella spp.

Preferably, the disease or disorder associated with intestinal dysbiosis is IBD, more preferably the IBD is ulcerative colitis or Crohn's disease, most preferably ulcerative colitis.

Preferably the biological sample mentioned in the uses and methods according to the invention is a stool sample.

### BRIEF DESCRIPTION OF THE FIGURES

FIG. 1A: Succinate conversion by fecal microbiomes. Fecal samples of 10 adult healthy humans were diluted and in vitro tested for their ability to convert succinate into propionate after two and seven days. Each diagram corresponds to a distinct fecal microbiome, ranging from approximately 93 to 4500 viable cells. Succinate conversion was measured after two and seven days of anaerobic batch cultivation in Hungate tubes at 37° C., in a defined base medium containing 30 mM of succinate as main carbon source and buffered at a pH of 6.5. The figure shows the amount of succinate (y-axis) that was metabolized after a specific amount of time (x-axis).

FIG. 1B: Bacterial genera enriched during in vitro enrichments. The three most strongly enriched genera in the tested fecal microbiomes were Phascolarctobacterium, Phascolarctobacterium_A, and Dialister. Each symbol shows the increase in relative abundance of the three genera corrected for background growth. Circles are measurements after two days, triangles are measurements after 7 days. Missing symbols indicate that the genus was not detected in a sample. The dashed vertical line indicates a 10% succinate-specific increase.

FIG. 2: Succinotype distributions in healthy individuals and IBD patients. The ratio of Dialister to Phascolarctobacterium abundance shows a strong bimodal distribution, implying that individuals can be classified into one of two ‘succinotypes’: D-type (Dialister dominance) or P-type (Phascolarctobacterium dominance). Microbiomes of healthy individuals can be of either of the D-type or of the P-type. P-types, however, are more prevalent. IBD patient cohorts were analyzed. Microbiomes from individuals suffering from IBD (UC or CD) were significantly more likely to be D-type.

FIG. 3: Transition probability of succinotype assigned in human fecal microbiomes. Human fecal microbiomes with longitudinal samples were classified into either ‘D’ or ‘P’ succinotypes based on their relative abundance of Dialister and Phascolarctobacterium. The numbers show the fraction of samples that maintained or changed their succinotype over time. Individuals typically maintain their succinotype over time. The probability that an individual maintains her or his succinotype between two fecal samples in the UCC cohort is 0.91 and 0.92 for C and P, respectively.

FIGS. 4A and 4B: The overall relative abundance of succinate-utilizing bacteria is not sufficient to distinguish between healthy individuals and IBD patients (FIG. 4A), only the Dialister/Phascolarctobacterium ratio is able to differentiate between the healthy and IBD cohort (FIG. 4B). The overall relative abundance was measured as the sum of relative abundances of Phascolarctobacterium and Dialister. H: healthy, UC: ulcerative colitis, CD: Crohn's disease. UC and CD patients were significantly more likely to have a D-succinotype microbiome than a P-succinotype one.

The Dialister/Phascolarctobacterium ratio revealed that individuals with IBD are significantly more likely to have a Dialister succinotype than a Phascolarctobacterium succinotype. Generalized mixed effects logistic regression n=7446, cohorts=7, UC vs. Healthy: p=0.000862, CD vs. Health: p=0.00107.

FIG. 5: Total relative abundance of succinate consumers Dialister and Phascolarctobacterium in human fecal microbiomes. The sum of relative abundances of Dialister and Phascolarctobacterium (sum of D and P) does not differ significantly between healthy individuals and UC or Crohn's disease patients.

FIG. 6A: Succinate utilisation rates of four succinate-utilizing species. Succinate utilisation rates of four succinate-utilizing species were determined. The symbols show the amount of succinate (y-axis) that was metabolized by the bacteria after a specific amount of time (x-axis). Phascolarctobacterium faecium (two proprietary isolates; A) and Phascolarctobacterium_A succinatutens (B) utilize succinate at a faster rate as Dialister hominis (C) and Dialister invisus (D). Circles and triangles indicate biological replicates. The utilization rate was computed from the slope of a linear fit to the utilization curve.

FIG. 6B: Estimated succinate consumption rate of bacterial isolates from the genera Phascolarctobacterium, Phascolarctobacterium_A, and Dialister. The circles show the conversion rate and the error bars the confidence intervals estimated by linear regression of the data from FIG. 4A. A) Phascolarctobacterium faecium, B) Phascolarctobacterium_A succinatutens, C) Dialister hominis, D) Dialister invisus.

FIGS. 7A and 7B: Dialister/Phascolarctobacterium ratio is indicative for a range of diseases and physical conditions, including IBD, colorectal cancer (CRC), atherosclerotic cardiovascular disease (ACVD) and low body mass index (BMI). (A) The points show the change in log-odds of disease when having a Phascolarctobacterium succinotype compared to a Dialister succinotype. Estimates were obtained from a generalized linear model with logit link function. The data used was obtained from the curatedMetagenomicData public dataset. m shows the number of studies used in the analysis of each disease, and n are the total number of patients or healthy individuals used in the respective analysis. The error bars show the estimated confidence interval. (B) The data points show the log-odds of being of Phascolarctobacterium succinotype for an individual of normal/overweight and underweight BMI (FIG. 7B).

FIG. 8: The informative power of the D/P biomarker can be improved by testing whether a subgroup of succinotypes has a microbiome comprising strains of the Prevotella genus. Individuals with a Dialister succinotype are more likely to have IBD than individuals with a Phascolarctobacterium succinotype in the analyzed cohort. Combining the presence of detectable Prevotella with the stratification based on succinotype further increases the proportion of individuals that have IBD.

### DETAILED DESCRIPTION OF THE INVENTION

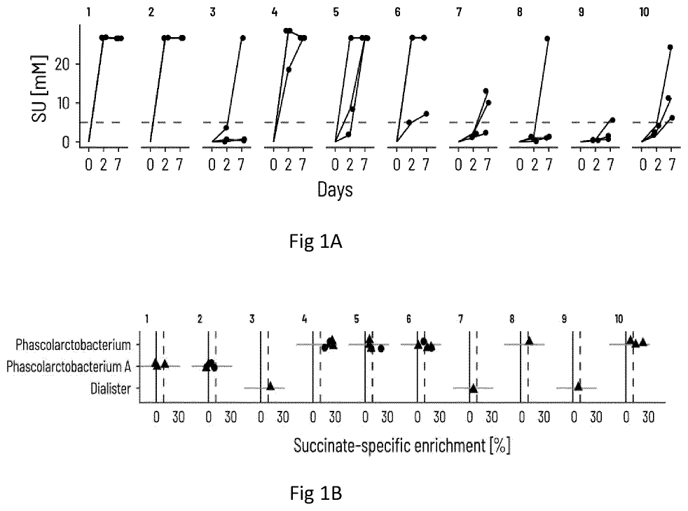

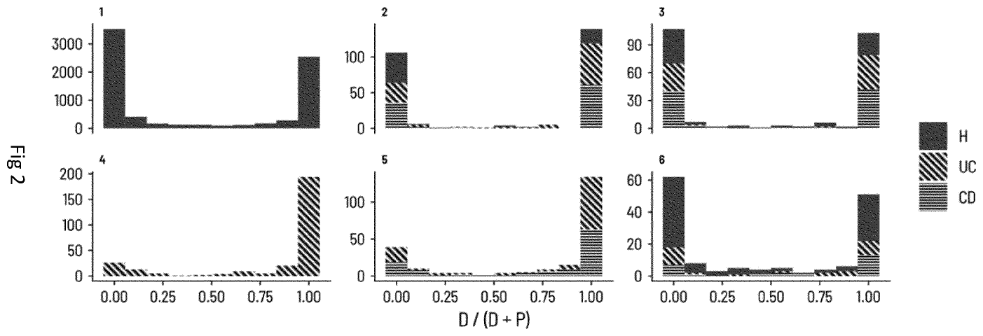

The inventors characterized the distribution of succinate-utilizing bacteria in healthy human microbiomes and determined whether the various succinate-metabolizing bacteria mechanistically differ in their ability to metabolize succinate (FIG. 1).

The inventors further determined the putative dominant succinate-utilizing taxon in the microbiomes of different individuals. For each of these putative utilizers as well as commonly mentioned putative utilizers in the literature, their ability to metabolize extracellular succinate in single culture assays was either confirmed or rejected. Using this approach, led to the identification of Dialister and Phascolarctobacterium as the likely most relevant succinate utilizers in human microbiomes.

With this mechanistic understanding of microbial succinate utilization in the human intestine at hand, the inventors next investigated how Dialister and Phascolarctobacterium are distributed across healthy microbiomes. The inventors analyzed data from more than 7000 individuals of the American Gut Project who report not to suffer from IBD. The ratio of Dialister to Phascolarctobacterium abundance shows a strong bimodal distribution (FIG. 2 (1)) implying that individuals can be classified into one of two ‘succinotypes’: D-type (Dialister dominance) or P-type (Phascolarctobacterium dominance). Both succinotypes are found in healthy individuals, although P-types are more prevalent.

Further analysis of IBD patient cohorts revealed that microbiomes from individuals suffering from IBD were significantly more likely to be D-type, with P-type being the rare exception among IBD patients (FIG. 2 (2)-(6)).

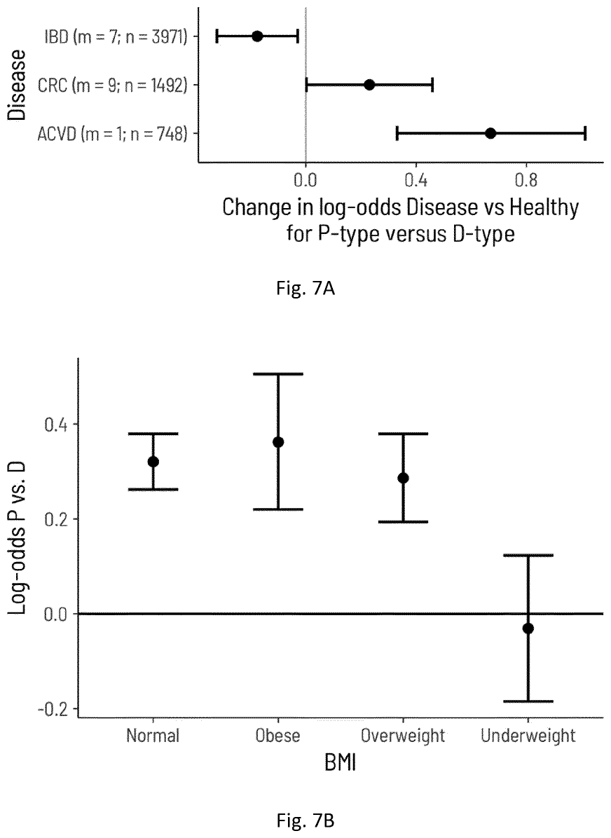

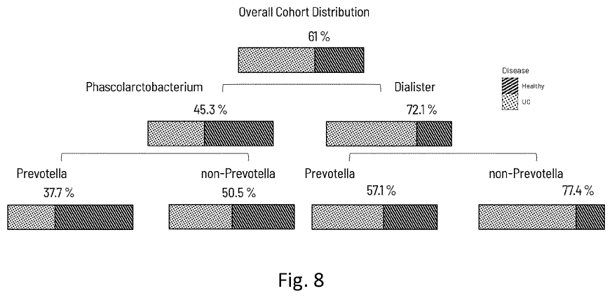

Importantly, the succinotyping approach (i.e., the determination of a subject's microbiome succinotype based on the determination of the identity of the dominant succinate-utilizing bacterium in said microbiome) based on the relative ratio of Dialister and Phascolarctobacterium was able to significantly differentiate between diseased and healthy microbiomes, while in contrast, simply quantifying the general abundance of succinate-utilizing bacteria was not able to differentiate between the microbiome of a healthy individual from a range of diseases and physical conditions, including IBD, colorectal cancer (CRC), atherosclerotic cardiovascular disease (ACVD) and underweight (low body mass index; BMI); (FIGS. 4 and 7).

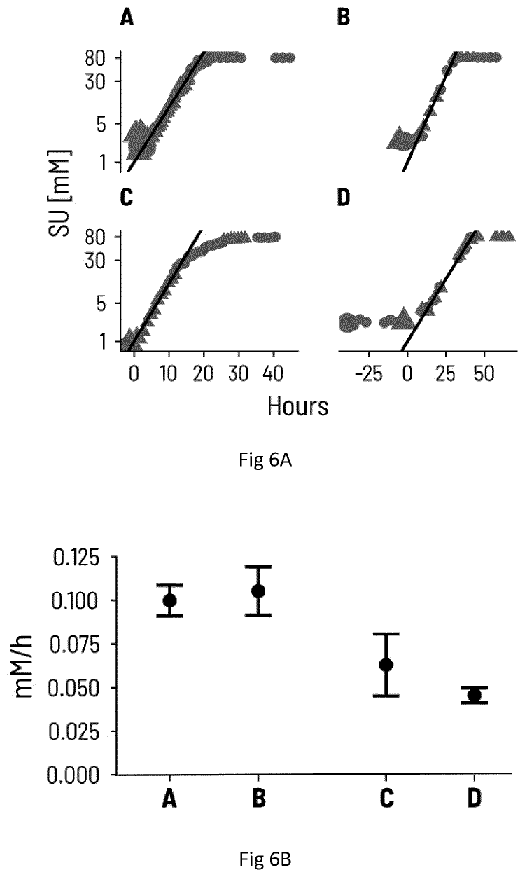

This implies that the identity of the dominant succinate-utilizing bacterium in a microbiome is an important contributing biomarker for such diseases and disorders. Based on this insight, the inventors revisited their in vitro characterization of the succinate-metabolic capabilities of these bacteria and found that Phascolarctobacterium spp. could metabolize succinate at twice the rate as Dialister sp. (FIGS. 6A and 6B).

This kinetic differentiation between the two succinotypes thus creates a mechanistic link between the significant association of D-type and IBD, CRC, ACVD and BMI and the body of literature that implicates microbiome succinate homeostasis in inflammatory intestinal disease.

### Definition

By “Dialister spp.” is intended to refer herein to a genus of Firmicutes bacteria classified within the class Negativicutes. The currently accepted taxonomy is based on the List of Prokaryotic names with Standing in Nomenclature (LPSN) under Record number: 515522 and on National Center for Biotechnology Information (NCBI) under Taxonomy ID: 39948. The Dialister spp. is defined by a bacterium having a 16S rRNA sequence having at least 85%, at least 90%, at least 95%, at least 99% or at least 100% identity with any one of SEQ ID Nos: 1, 2, 3, 4, 5, 6 and 7.

By “Phascolarctobacterium spp.” is intended to refer herein to a genus of Firmicutes bacteria classified within the class Negativicutes. The currently accepted taxonomy is based on the List of Prokaryotic names with Standing in Nomenclature (LPSN) under Record number: 516310 and on National Center for Biotechnology Information (NCBI) under Taxonomy ID: 33024. The Phascolarctobacterium spp. is defined by a bacterium having a 16S rRNA sequence having at least 85%, at least 90%, at least 95%, at least 99% or at least 100% identity with any one of SEQ ID Nos: 8, 9 and 10.

The abbreviation “spp.” indicates species pluralis (i.e., “several species”) refers to the whole genus of bacteria, to several or all species of the genus.

The term “16S rRNA gene” as used herein, refers to a bacterial gene encoding the component of the 30S small subunit of a prokaryotic ribosome that binds to the Shine-Dalgamo sequence. Sequence analysis of the 16S ribosomal RNA (rRNA) gene has been widely used to identify bacterial species and perform taxonomic studies. Bacterial 16S rRNA genes generally contain nine “hypervariable regions” that demonstrate considerable sequence diversity among different bacterial species and can be used for species identification. Thus, the term “hypervariable regions of the 16S rRNA gene”, as used herein, refers to said sequences in the 16S ribosomal rRNA gene, that allow identifying a single bacterial species or differentiating among a limited number of different species or genera.

The expression “ratio of Dialister spp. to Phascolarctobacterium spp.” or “D/P ratio”, as used herein, refers to the result of dividing the total number, percentage, abundance or concentration of Dialister spp. by the total number, percentage, abundance or concentration of Phascolarctobacterium spp., respectively. The ratio is also meant to cover any mathematically equivalent function or isomorphism, in particular
the inverse ratio, i.e., the ratio of Phascolarctobacterium spp. to Dialister spp. (e.g., ratio=P/D),
the ratio of the logarithm of the total number, percentage, abundance, or concentration of Dialister and Phascolarctobacterium, respectively (e.g., ratio=log (D)−log (P)),
the relative proportion of Dialister and Phascolarctobacterium (e.g., Ratio=D/(D+P)).

Thus, when the ratio of Dialister spp. to Phascolarctobacterium spp. is mentioned in the present application, it could be replaced by the ratio of Phascolarctobacterium spp. to Dialister spp. in any use or method of the present invention. In the case, when a ratio varies, it is the opposition for this ratio. For instance, when the ratio of Dialister spp. to Phascolarctobacterium spp. increases, the ratio of Phascolarctobacterium spp. to Dialister spp. decreases and on the opposite, when the ratio of Dialister spp. to Phascolarctobacterium spp. decreases, the ratio of Phascolarctobacterium spp. to Dialister spp. increases.

In some aspect, the ratio of Dialister spp. to Phascolarctobacterium spp. (D/P) is a ratio comparing:
(i) Dialister hominis and (ii) Phascolarctobacterium faecium;
(i) Dialister hominis and (ii) Phascolarctobacterium_A succinatutens;
(i) Dialister hominis and (ii) Phascolarctobacterium faecium and Phascolarctobacterium_A succinatutens;
(i) Dialister invisus and (ii) Phascolarctobacterium faecium;
(i) Dialister invisus and (ii) Phascolarctobacterium_A succinatutens;
(i) Dialister invisus and (ii) Phascolarctobacterium faecium and Phascolarctobacterium_A succinatutens;
(i) Dialister hominis and Dialister invisus and (ii) Phascolarctobacterium faecium and Phascolarctobacterium_A succinatutens;
(i) Dialister invisus and Dialister hominis and (ii) Phascolarctobacterium faecium; or
(i) Dialiser invisus and Dialister hominis and (ii) Phascolarctobacterium succinatutens.

As used herein, the terms “Dialister succinotype”, “D-type” and “Dialister dominance” are used interchangeably and refer to a condition in which the bacteria of the genus Dialister are more represented than bacteria from the genus Phascolarctobacterium, in particular a condition in which the ratio D/P is superior to 1 (or conversely a P/D ratio inferior to 1). A subject having a Dialister succinotype or being of D-type thus corresponds to a subject that has more bacteria from the genus Dialister than from the Phascolarctobacterium genus in a microbiome, preferably in the intestinal microbiome, of said subject, preferably a D/P ratio superior to 1, (or conversely a P/D ratio inferior to 1), in particular in a microbiome from a stool sample from said subject, that reflects the intestinal microbiome of said subject.

As used herein, the terms “Phascolarctobacterium succinotype”, “P-type” and “Phascolarctobacterium dominance” are used interchangeably and refer to a condition in which the bacteria of the genus Phascolarctobacterium are more represented than bacteria from the genus Dialister, in particular a condition in which the ratio P/D ratio is superior to 1 (or conversely a D/P ratio inferior to 1). A subject having a Phascolarctobacterium succinotype or being of P-type thus corresponds to a subject that has more bacteria from the Phascolarctobacterium genus than from the Dialister genus in a microbiome, preferably in the intestinal microbiome, of said subject, preferably a P/D ratio superior to 1 (or conversely a D/P ratio inferior to 1), in particular in a microbiome from a stool sample from said subject, that reflects the intestinal microbiome of said subject.

The expression “stool”, as used herein, refers to the solid or semisolid fecal remains of food that could not be digested in the intestine. In a particular embodiment, a stool sample is collected from a subject after defecation. The microbiome content of a stool sample from a subject particularly reflects the intestinal microbiome of said subject.

The term “subject”, “individual” or “patient”, as used herein, refers to all animals classified as mammals and includes, but is not restricted to, domestic and farm animals, primates and humans, e.g., human beings, non-human primates, cows, horses, pigs, sheep, goats, dogs, cats or rodents. Preferably, the subject is a male or female human.

The term “primer set”, as used herein, refers to a set of oligonucleotides of RNA or DNA (preferably of about 15-35 bases) that specifically hybridizes to the hypervariable regions of the 16S rRNA gene and serves as a starting point for DNA synthesis. They are required for DNA amplification mediated by a DNA polymerase in a reaction based on the PCR technique. The relative amount, concentration and/or average size of each amplicon can then be analyzed with techniques known by the person skilled in the art. Non-limiting examples of such techniques are gel electrophoresis, or techniques based on the RT-PCR technique. It is also possible to sequence the target nucleic acid using said primers and after further steps known to the expert in the field.

The term “probe”, as used herein, refers to DNA or RNA oligonucleotide sequences that hybridize by complementarity with a specific sequence. In other words, the probe hybridizes to specific single-stranded nucleic acid (DNA or RNA) whose base sequence allows probe-target base pairing due to complementarity between the probe and the target. In a preferred aspect, the subsequent hybrid can be detected using techniques known by the expert in the field. For instance, the probe can be labelled with a marker that can be radioactive or (a) fluorescent molecule(s) and immobilized on a membrane or in situ. Commonly used markers are ³²P (a radioactive isotope of phosphorus incorporated into the phosphodiester bond in the probe DNA) or Digoxigenin, which is a non-radioactive, antibody-based marker. DNA sequences or RNA transcripts that have moderate to high sequence similarity to the probe are then detected by visualizing the hybridized probe via autoradiography or other imaging techniques. Normally, either X-ray pictures are taken of the filter, or the filter is placed under UV light, or under a microscope for the detection of the fluorescently labelled probe. Detection of sequences with moderate or high similarity depends on how stringent the hybridization conditions were applied-high stringency, such as high hybridization temperature and low salt in hybridization buffers, permits only hybridization between nucleic acid sequences that are highly similar, whereas low stringency, such as lower temperature and high salt, allows hybridization when the sequences are less similar.

The term “oligonucleotide”, as used herein, refers to a single-stranded DNA or RNA molecule, preferably with up to 35, 30, 25, 20, 19, 18, 17, 16, 15, 14 or 13 bases in length (upper limit). The oligonucleotides of the invention are DNA or RNA molecules, preferably of at least 2, at least 5, at least 10, at least 12, at least 15, at least 16, at least 17, at least 18, at least 19, at least 20, at least 21, at least 22, at least 23, at least 25 nucleotide bases in length (lower limit). Ranges of base lengths can be combined in all different manners using the afore-mentioned lower and upper limits, for example at least 2 and up to 30 bases, at least 10 and up to 15 bases, at least 5 and up 15 bases or at least 15 and up to 18 bases.

The term “specifically hybridize”, as used herein, refers to the conditions which allow the hybridization of two polynucleotides under high stringent conditions or moderately stringent conditions. The “stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and the target sequence, the higher the relative temperature which must be used. As a result, it follows that higher relative temperatures would tend to make the reaction conditions more stringent, while lower temperatures less so.

As used herein, the term “hybridizing conditions” is intended to mean those conditions of time, temperature, and pH, and the necessary amounts and concentrations of reactants and reagents, sufficient to allow at least a portion of complementary sequences to anneal with each other. As it is well known in the art, the time, temperature, and pH conditions required to accomplish hybridization depend on the size of the oligonucleotide probe or primer to be hybridized, the degree of complementarity between the oligonucleotide probe or primer and the target, the nucleotide type (e.g., RNA, or DNA) of the oligonucleotide probe or primer and the target, and the presence of other materials in the hybridization reaction mixture. The actual conditions necessary for each hybridization step are well known in the art or can be determined without undue experimentation. General parameters for specific (i.e., stringent) hybridization conditions for nucleic acids are described in Sambrook, et al., Molecular Cloning: A Laboratory Manual (3rd Edition, 2001). One of skills in the art will in particular appreciate that as the oligonucleotides become shorter, it may become necessary to adjust their length to achieve a relatively uniform melting temperature for satisfactory hybridization results.

The terms “low stringency”, “medium stringency”, “medium/high stringency”, “high stringency” and “very high stringency” refer to conditions of hybridization. Suitable experimental conditions for determining hybridization between a nucleotide probe and a homologous DNA or RNA sequence involves presoaking of the filter containing the DNA fragments or RNA to hybridize in 5×SSC (Sodium chloride/Sodium citrate for 10 min, and prehybridization of the filter in a solution of 5×SSC, 5×Denhardt's solution, 0.5% SDS and 100 μg/ml of denatured sonicated salmon sperm DNA, followed by hybridization in the same solution containing a concentration of 10 ng/ml of a random-primed 32P-dCTP-labeled (specific activity >1×109 cpm/μg) probe for 12 hours at ca. 45° C. (Feinberg and Vogelstein, 1983). For various stringency conditions the filter is then washed twice for 30 minutes in 2×SSC, 0.5% SDS and at least 55° C. (low stringency), more preferably at least 60° C. (medium stringency), still more preferably at least 65° C. (medium/high stringency), even more preferably at least 70° C. (high stringency), and even more preferably at least 75° C. (very high stringency).

As used herein, the term “sequence identity” or “identity” refers to the number (%) of matches (identical amino acid residues) in positions from an alignment of two polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithm (e.g., Needleman and Wunsch algorithm; Needleman and Wunsch, 1970) which aligns the sequences optimally over the entire length, while sequences of substantially different lengths are preferably aligned using a local alignment algorithm (e.g., Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://blast.ncbi.nlm.nih.gov/or http://www.ebi.ac.uk/Tools/emboss/). Those skilled in the art can determine appropriate parameters for measuring alignment, including any algorithms needed to achieve maximal alignment over the full length of the sequences being compared. Preferably, for purposes herein, % amino acid sequence identity values refers to values generated using the pair wise sequence alignment program EMBOSS Needle that creates an optimal global alignment of two sequences using the Needleman-Wunsch algorithm, wherein all search parameters are set to default values, i.e., Scoring matrix=BLOSUM62, Gap open=10, Gap extend=0.5, End gap penalty=false, End gap open=10 and End gap extend=0.5.

The terms “microbiome” and “microbiota” as used herein are known as synonyms and particularly denote the totality of microbial life forms within a given habitat or host. The microbiome can be for example an intestinal microbiome, a fecal microbiome, a mouth or nasal microbiome, a vaginal microbiome, a skin microbiome, a lung microbiome, a waste-treatment microbiome, a soil microbiome, a plant-associated microbiome or a microbiome used for food fermentation. The term “intestinal microbiome” in particular refers to the gut microbiota.

The “relative abundance” is a component of biodiversity and refers to how common or rare a bacterial population is relative to other microbe populations. Relative abundance is preferably the percent composition of a bacterial population relative to the total number of microbes. The relative abundance of a particular bacterial population is calculated by measuring a proxy for abundance (e.g., a number of sequencing amplicon reads mapped to a gene, a genome coverage . . . ) and by dividing the measured quantity of the particular bacterial population by the sum of the measured quantities across all the microbiome.

The term “treatment” refers to any act intended to ameliorate the health status of patients such as therapy, prevention, prophylaxis and retardation of the disease or of the symptoms of the disease. It designates both a curative treatment and/or a prophylactic treatment of a disease. A curative treatment is defined as a treatment resulting in cure or a treatment alleviating, improving and/or eliminating, reducing and/or stabilizing a disease or the symptoms of a disease or the suffering that it causes directly or indirectly. A prophylactic treatment comprises both a treatment resulting in the prevention of a disease and a treatment reducing and/or delaying the progression and/or the incidence of a disease or the risk of its occurrence. In certain embodiments, such a term refers to the improvement or eradication of a disease, a disorder, an infection or symptoms associated with it. In particular, the disease is an intestinal dysbiosis optionally associated with IBD, ulcerative colitis or Crohn's disease. Treatments according to the present invention do not necessarily imply 100% or complete treatment. Rather, there are varying degrees of treatment of which one of ordinary skill in the art recognizes as having a potential benefit or therapeutic effect.

As used herein, the terms “disorder” or “disease” refer to the incorrectly functioning organ, part, structure, or system of the body resulting from the effect of genetic or developmental errors, infections, poisons, nutritional deficiency or imbalance, toxicity, or unfavorable environmental factors. Preferably, these terms refer to a health disorder or disease e.g., an illness that disrupts normal physical or mental functions. Preferably, the disease is caused by an intestinal dysbiosis, in particular preferably IBD, ulcerative colitis or Crohn's disease.

The term “dysbiosis” Is known and denotes the alteration of the microbiota in comparison to the healthy state. The microbiota's state may be characterized by determining key markers, intermediate metabolites and end metabolites. A healthy microbiota can be characterized by the presence or absence of certain bacteria gender or by the absence of intermediate metabolites. In the context of the present invention, the dysbiosis is an intestinal dysbiosis.

Within the context of this invention, “responder”, “responsive” or “have a therapeutic benefit” refers to a subject who responds to a treatment, e.g., at least one of the symptoms is alleviated, or the development of the disease is stopped, or slowed down. Typically, a subject who responds to a treatment is a subject who will be completely treated (cured), e.g., a subject who will be cured or prevented to suffer from dysbiosis and/or IBD. By “susceptible to have a therapeutic benefit” is intended a patient who shows a good therapeutic benefit of the treatment, that is to say cure of the disease, alleviated symptoms, progression of the disease which is stopped or slowed down, an improved health condition, an improved live comfort, a longer disease-free survival, a longer overall survival in comparison to a population of patients suffering from the same disease and having the same treatment.

As used herein, a “pharmaceutical composition” refers to a preparation of one or more of the active agents, such as comprising viable Phascolarctobacterium spp. bacteria, with optional other chemical components such as physiologically suitable carriers and excipients. Compositions of the present invention can be in a form suitable for any conventional route of administration or use. In one embodiment, a “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. An “acceptable vehicle” or “pharmaceutically acceptable carrier” as referred to herein, is any known compound or combination of compounds that are known to those skilled in the art to be useful in formulating pharmaceutical compositions.

Ratio of Dialister Spp. To Phascolarctobacterium Spp. And Uses Thereof

The ratio of Dialister spp. to Phascolarctobacterium spp. (or the ratio of Phascolarctobacterium spp. to Dialister spp.) has been identified by the inventors as being indicative of an intestinal dysbiosis or of a disease or disorder that is associated with an intestinal dysbiosis.

In particular, the ratio of Dialister spp. to Phascolarctobacterium spp. has been identified by the inventors as being indicative of Inflammatory Bowel Disease (IBD), including ulcerative colitis or Crohn's disease, colorectal cancer (CRC), atherosclerotic cardiovascular disease (ACVD), and underweight (low body mass index).

The ratio of Dialister spp. to Phascolarctobacterium spp. is determined by any of the techniques available and known by the person skilled in the art.

The ratio of Dialister spp. to Phascolarctobacterium spp. is determined in a sample from an individual, preferably from a biological sample of said individual, especially a stool sample. Such ratio can be determined in an individual or in a group of individuals.

The present invention relates to a method for determining the ratio of Dialister spp. to Phascolarctobacterium spp. in a sample, preferably a biological sample, even more preferably a stool sample, from an individual or a group of individuals, the method comprising
determining the amount of Dialister spp. in a biological sample, preferably a stool sample of said individual or group of individuals;
determining the amount of Phascolarctobacterium spp. in a biological sample, preferably a stool sample of said individual; and
dividing the amount of Dialister spp. by the amount of Phascolarctobacterium spp., thereby determining the ratio of Dialister spp. to Phascolarctobacterium spp. in the sample.

The method may further comprise a previous step of providing a sample from the individual, preferably a biological sample, especially a stool sample.

By amount is intended any means allowing to assess the total number, percentage, abundance or concentration of bacteria.

The abundance can be for instance measured using optical density, qPCR, flow cytometry, microarray technique, amplicon sequencing, single cell sequencing or single cell Matrix Assisted Laser Desorption Ionization—Time of Flight (MALDI-TOF) following flow cytometry or other microfluidic methods, chamber counting, total bacterial DNA quantification or metagenomic sequencing and grouping of genes, proteomic profiling, total RNA sequencing of complex samples. These methods are well known by the man skilled in the art.

In a preferred aspect, the abundance is measured by amplifying the V3/V4 region of the hypervariable regions of the 16S rRNA gene and by sequencing amplicons. An example of primer sets specific for Dialister spp., e.g., for performing a qPCR analysis, is the following: 5′-GAGTATCGGAGAGGAAAGTGGA-3′ (SEQ ID No: 11) and 5′-GCTTCTCTTTGTTGACACCCATT-3′ (SEQ ID No: 12). An example of primer sets specific for Phascolarctobacterium spp. is the following: 5′-GGAGTGCTAATACCGGATGTGA-3′ (SEQ ID No: 13) and 5′-CCGTGGCTTCCTCGTTTACT-3′ (SEQ ID No: 14).

In a particular aspect, the abundance is the relative abundance and the ratio is determined by dividing the relative abundance of Dialister spp. by the relative abundance of Phascolarctobacterium spp.

In a particular aspect, the hypervariable regions of the 16S rRNA gene can be used to identify or differentiate Dialister spp. and Phascolarctobacterium spp. The identification of said regions can be mediated by techniques well known by the person skilled in the art. Non-limiting examples of such techniques are polymerase chain reaction (PCR) amplification, Real Time polymerase chain reaction (RT-PCR), In situ Hybridization (ISH), Northern blot or Micro-array, sequencing.

More particularly, the hypervariable regions of the 16S rRNA gene are used to identify bacteria of the species Dialister spp. and Phascolarctobacterium spp.

This ratio can be used as a biomarker. In particular, it can be used as a biomarker, in particular of intestinal dysbiosis or of a disorder or disease associated with intestinal dysbiosis, for diagnosis purposes, for treatment response monitoring purposes, for disease course monitoring purposes, for selecting a subject that will benefit from a treatment, or for stratifying patient population in a clinical trial.

In an embodiment, the D/P ratio is for use as a biomarker for diagnosis purpose of a disease or disorder selected from the group consisting of IBD, colorectal cancer, atherosclerotic cardiovascular disease and low body mass index (underweight), preferably IBD. This biomarker provides information useful for assessing whether an individual is suffering or is at risk of developing such disease or disorder.

Especially, this ratio can be used as a biomarker for diagnosis purposes of IBD, including ulcerative colitis and Crohn's disease. In addition, the ratio can be used as biomarker for treatment response monitoring purposes, in particular treatment of IBD, including ulcerative colitis and Crohn's disease; for disease course monitoring purposes, in particular IBD course and stages, IBD being optionally ulcerative colitis and Crohn's disease; or for stratifying patient population in a clinical trial, in particular a clinical trial including IBD subjects, optionally subjects suffering of ulcerative colitis and Crohn's disease, the clinical trials of candidate medicament for the treatment of IBD.

This biomarker provides information useful for assessing whether an individual is suffering of IBD or is at risk of developing IBD. The IBD can be more specifically ulcerative colitis or Crohn's disease.

In an embodiment, the biomarker is a composite biomarker comprising:
the ratio of Dialister spp. to Phascolarctobacterium spp. (D/P) or the ratio of Phascolarctobacterium spp. to Dialister spp. (P/D); and
the presence or absence of Prevotella spp.

The determination of the presence or absence of Prevotella spp, particularly increases the accuracy of the ratio D/P or P/D biomarker.

In some embodiments, the presence of Prevotella spp. is an amount or a relative abundance of Prevotella in the microbial population, in particular a microbial population from a biological sample. The presence of Prevotella spp. is particularly a relative abundance of Prevotella spp. superior to 0.01%.

In some embodiments, the absence of Prevotella spp. is an amount or a relative abundance of Prevotella in the microbial population, in particular a microbial population from a biological sample. The absence of Prevotella spp. is particularly a relative abundance of Prevotella spp. inferior to 0.01%.

Accordingly, the present invention relates to a method for assessing whether an individual is suffering or is at risk of developing a disease or disorder associated with an intestinal dysbiosis, preferably IBD, or for providing information useful for assessing whether an individual is suffering or is at risk of developing a disease or disorder associated with an intestinal dysbiosis, preferably IBD, the method comprising:
a) providing a biological sample from the individual;
b) determining a ratio of Dialister spp. to Phascolarctobacterium spp. in the biological sample; and,
c) classifying the individual as suffering or being at risk of developing a disease or disorder associated with an intestinal dysbiosis based on the determined ratio of Dialister spp. to Phascolarctobacterium spp.

Preferably, the biological sample is a stool sample.

Optionally, a D/P ratio higher than 1 (i.e., D/P>1) is indicative of an individual suffering or being at risk of developing a disease or disorder associated with an intestinal dysbiosis preferably IBD or underweight (BMI<18.5). Particularly, a D/P ratio higher than 1.05, 1.1, 1.2 or 1.3 is indicative of an individual suffering or being at risk of developing a disease or disorder associated with an intestinal dysbiosis, preferably IBD.

Alternatively, a ratio inferior than 1 (i.e., D/P<1) is indicative of an individual suffering or being at risk of developing a disease or disorder associated with an intestinal dysbiosis such as colorectal cancer and atherosclerotic cardiovascular disease. Optionally, a D/P ratio lower than 1 (i.e., D/P<1) is indicative of an individual suffering or being at risk of developing a disease or disorder associated with an intestinal dysbiosis such as colorectal cancer and atherosclerotic cardiovascular disease. Optionally, a D/P ratio lower than 0.95, 0.9, 0.8 or 0.7 is indicative of an individual suffering or being at risk of developing a disease or disorder associated with an intestinal dysbiosis such as colorectal cancer and atherosclerotic cardiovascular disease. In particular, the D/P ratio is comprised between 0.1 and 0.95, between 0.1 and 0.9, between 0.1 and 0.7 or between 0.1 and 0.5.

Optionally, the method may further comprise the determination of the presence or absence of Prevotella spp. In particular, the absence of Prevotella spp. increases the probability that the individual is suffering or is at risk of developing a disease or disorder associated with an intestinal dysbiosis.

Preferably, the disease or disorder associated with an intestinal dysbiosis is inflammatory bowel disease (IBD), and the method further comprises the determination of the presence or absence of Prevotella spp. in the biological sample, wherein the subject is classified as suffering or being at risk of developing inflammatory bowel disease (IBD) if the ratio D/P is superior to 1, and Prevotella is absent.

Optionally, when the D/P ratio is superior to 1 the method may further comprise a step of administering a treatment, in particular a veterinary or pharmaceutical composition comprising Phascolarctobacterium spp., preferably Phascolarctobacterium faecium and/or Phascolarctobacterium_A succinatutens and optionally Prevotella spp. or a combined preparation for simultaneous, separate or sequential use comprising i) an agent able to decrease Dialister spp. population in a subject and ii) an agent able to increase Phascolarctobacterium spp. population in a subject.

In a particular embodiment, the present invention relates to a method for assessing whether an individual is suffering or is at risk of developing inflammatory bowel disease (IBD) or for providing information useful for assessing whether an individual is suffering or is at risk of developing inflammatory bowel disease (IBD), the method comprising:
a) providing a stool sample from the individual;
b) determining a ratio of Dialister spp. To Phascolarctobacterium spp. in the stool sample; and,
c) classifying the individual as suffering or being at risk of developing inflammatory bowel disease (IBD) based on the determined ratio of Dialister spp. to Phascolarctobacterium spp.

Optionally, a ratio higher than 1 (e.g., D/P>1) is indicative of an individual as suffering or being at risk of developing inflammatory bowel disease (IBD). Optionally, a ratio higher than 1.05, 1.1, 1.2 or 1.3 is indicative of an individual as suffering or being at risk of developing inflammatory bowel disease (IBD). In particular, the D/P ratio is comprised between 1 and 5, between 1 and 4, between 1 and 3 or between 1 and 2. Optionally, the method may further comprise the determination of the presence or absence of Prevotella spp. In particular, the absence of Prevotella spp. increases the probability that the individual is suffering or is at risk of developing IBD.

Optionally, the method may further comprise a step of administering a treatment, in particular a veterinary or pharmaceutical composition comprising Phascolarctobacterium spp. and optionally Prevotella spp. or a combined preparation for simultaneous, separate or sequential use comprising i) an agent able to decrease Dialister spp. population in a subject and ii) an agent able to increase Phascolarctobacterium spp. population in a subject.

Alternatively, the present invention relates to a method for assessing whether an individual is suffering or is at risk of developing a disease or disorder associated with an intestinal dysbiosis or for providing information useful for assessing whether an individual is suffering or is at risk of developing a disease or disorder associated with an intestinal dysbiosis, the method comprising
a) providing a biological sample from the individual;
b) determining a ratio of Phascolarctobacterium spp. to Dialister spp. in the biological sample; and
c) classifying the individual as suffering or being at risk of developing a disease or disorder associated with an intestinal dysbiosis based on the determined ratio of Phascolarctobacterium spp. to Dialister spp.

Preferably the biological sample is a stool sample.

In an embodiment, a P/D ratio higher than 1 (i.e., P/D>1) is indicative of an individual suffering or being at risk of developing a disease or disorder associated with an intestinal dysbiosis such as colorectal cancer and atherosclerotic cardiovascular disease. Particularly, a P/D ratio higher than 1.05, 1.1, 1.2 or 1.3 is indicative of an individual suffering or being at risk of developing a disease or disorder associated with an intestinal dysbiosis such as colorectal cancer and atherosclerotic cardiovascular disease.

In another embodiment, a ratio lower than 1 (i.e., P/D<1) is indicative of an individual suffering or being at risk of developing a disease or disorder associated with an intestinal dysbiosis, preferably IBD.

Optionally, a ratio lower than 0.95, 0.9, 0.8 or 0.7 is indicative of an individual suffering or being at risk of developing a disease or disorder associated with an intestinal dysbiosis, preferably IBD. In particular, the P/D ratio is comprised between 0.1 and 0.95, between 0.1 and 0.9, between 0.1 and 0.7 or between 0.1 and 0.5.

Optionally, the method may further comprise the determination of the presence or absence of Prevotella spp. In particular, the absence of Prevotella spp. increases the probability that the individual is suffering or is at risk of developing a disease or disorder associated with an intestinal dysbiosis, preferably IBD.

Preferably, the disease or disorder associated with an intestinal dysbiosis is inflammatory bowel disease (IBD), and the method further comprises the determination of the presence or absence of Prevotella spp. in the biological sample, wherein the subject is classified as suffering or being at risk of developing inflammatory bowel disease (IBD) if the ratio P/D is inferior to 1, and Prevotella is absent.

Optionally, the method may further comprise a step of administering a treatment, in particular a veterinary or pharmaceutical composition comprising Phascolarctobacterium spp. and optionally Prevotella spp. or a combined preparation for simultaneous, separate or sequential use comprising i) an agent able to decrease Dialister spp. population in a subject and ii) an agent able to increase Phascolarctobacterium spp. population in a subject.

Particularly, the present invention relates to a method for assessing whether an individual is suffering or is at risk of developing inflammatory bowel disease (IBD) or for providing information useful for assessing whether an individual is suffering or is at risk of developing inflammatory bowel disease (IBD), the method comprising
a) providing a stool sample from the individual;
b) determining a ratio of Phascolarctobacterium spp. to Dialister spp. in the stool sample; and
c) classifying the individual as suffering or being at risk of developing inflammatory bowel disease (IBD) based on the determined ratio of Phascolarctobacterium spp. To Dialister spp.

Optionally, a ratio lower than 1 (i.e., P/D<1) is indicative of an individual suffering or being at risk of developing inflammatory bowel disease (IBD). Optionally, a ratio lower than 0.95, 0.9, 0.8 or 0.7 is indicative of an individual suffering or being at risk of developing inflammatory bowel disease (IBD). In particular, the P/D ratio is comprised between 0.1 and 0.95, between 0.1 and 0.9, between 0.1 and 0.7 or between 0.1 and 0.5.

Optionally, the method may further comprise a step of administering a treatment, in particular a veterinary or pharmaceutical composition comprising Phascolarctobacterium spp. and optionally Prevotella spp. or a combined preparation for simultaneous, separate or sequential use comprising i) an agent able to decrease Dialister spp. population in a subject and ii) an agent able to increase Phascolarctobacterium spp. population in a subject.

Optionally, the method may further comprise the determination of the presence or absence of Prevotella spp. In particular, the absence of Prevotella spp. increases the probability that the individual is suffering or is at risk of developing IBD.

In some embodiments, the methods disclosed herein may further comprise the steps of:
d) determining the presence or absence of Prevotella spp. in the biological sample from the subject, preferably the stool sample; and
e) combining the presence or absence of Prevotella spp. with the ratio of Dialister spp. to Phascolarctobacterium spp. (D/P) or Phascolarctobacterium spp. to Dialister spp. (P/D).

Preferably, if the ratio D/P is higher than 1 or the ratio P/D is lower than 1 and Prevotella is absent, the subject has higher probability of suffering or is even more at risk of developing the disease or disorder associated with an intestinal dysbiosis, preferably IBD.

In particular:
If the D/P ratio is higher than 1 or the P/D ratio is inferior to 1; and Prevotella spp. is absent, then the subject is at high risk of developing or suffering from a disease or disorder associated with an intestinal dysbiosis, preferably IBD; or
If the P/D ratio is higher than 1 or the D/P ratio is inferior to 1, and Prevotella spp. is present, then the subject has low risk of developing or suffering from a disease or disorder associated with an intestinal dysbiosis, preferably IBD.

In another aspect, the ratio of Dialister spp. to Phascolarctobacterium spp. is useful for disease or disorder course monitoring purposes. Particularly, the disease or disorder is a disease or disorder associated with an intestinal dysbiosis, preferably selected from the group consisting of IBD, including ulcerative colitis or Crohn's disease, colorectal cancer, atherosclerotic cardiovascular disease and low body mass index (underweight).

In some embodiments, the disease is IBD, especially ulcerative colitis or Crohn's disease. An increase of the ratio of Dialister spp. to Phascolarctobacterium spp. is indicative of a worsening of IBD, especially ulcerative colitis or Crohn's disease. Alternatively, a decrease of the ratio of Dialister spp. To Phascolarctobacterium spp. is indicative of an improvement or regression of IBD, especially ulcerative colitis or Crohn's disease.

In some embodiments, the disease is CRC or ACVD. An increase of the ratio of Phascolarctobacterium spp. to Dialister spp. is indicative of a worsening of said diseases. Alternatively, a decrease of the ratio of Phascolarctobacterium spp. To Dialister spp. is indicative of an improvement or regression of said diseases.

Accordingly, the present invention relates to a method for monitoring the course of a disease or disorder associated with an intestinal dysbiosis, preferably IBD, in a patient or for providing information useful for monitoring the course of a disease or disorder associated with an intestinal dysbiosis, preferably IBD, in a patient, the method comprising
a) providing a first biological sample, preferably a stool sample, from the individual at a first time;
b) determining a first ratio of Dialister spp. to Phascolarctobacterium spp. in the first biological sample;
c) providing a second biological sample, preferably a stool sample, from the individual at a second time, the second time being after the first time;
d) determining a second ratio of Dialister spp. to Phascolarctobacterium spp. in the second biological sample; and
e) comparing the first ratio and the second ratio.

Preferably, the disease or disorder associated with an intestinal dysbiosis is selected from the group consisting of IBD, particularly ulcerative colitis or Crohn's disease, colorectal cancer, atherosclerotic cardiovascular disease and low body mass index (underweight), preferably is IBD.

In an embodiment, the disease or disorder associated with an intestinal dysbiosis is selected from the group consisting of IBD, particularly ulcerative colitis or Crohn's disease, and low body mass index (underweight), preferably is IBD and an increase of the ratio is indicative of a worsening of the disease or disorder associated with an intestinal dysbiosis and/or a decrease of the ratio is indicative of an improvement or regression of the disease or disorder associated with an intestinal dysbiosis.

In particular, when the disease is IBD, especially ulcerative colitis or Crohn's disease, an increase of the ratio of Dialister spp. to Phascolarctobacterium spp. is indicative of a worsening of IBD, especially ulcerative colitis or Crohn's disease. Alternatively, a decrease of the ratio of Dialister spp. To Phascolarctobacterium spp. is indicative of an improvement or regression of IBD, especially ulcerative colitis or Crohn's disease.

Alternatively, when the disease is CRC or ACVD, an increase of the ratio of Dialister spp. to Phascolarctobacterium spp. is indicative of an improvement or regression of said diseases. Alternatively, a decrease of the ratio of Dialister spp. To Phascolarctobacterium spp. is indicative worsening of said diseases.

In another embodiment, the disease or disorder associated with an intestinal dysbiosis is selected from the group consisting of colorectal cancer and atherosclerotic cardiovascular disease, and a decrease of the ratio is indicative of a worsening of the disease or disorder associated with an intestinal dysbiosis and/or an increase of the ratio is indicative of an improvement or regression of the disease or disorder associated with an intestinal dysbiosis.

Preferably the biological sample is a stool sample.

Optionally, the method may further comprise the determination of the amount or relative abundance of Prevotella spp. in the first and second samples. In particular, a decrease of the amount or relative abundance of Prevotella spp. is indicative of a worsening of the disease or disorder associated with an intestinal dysbiosis; whereas an increase of the amount or relative abundance of Prevotella spp. is indicative of an improvement or regression of the disease or disorder associated with an intestinal dysbiosis.

In particular, the present invention relates to a method for monitoring the course of IBD in a patient or for providing information useful for monitoring the course of IBD in a patient, the method comprising
a) providing a first stool sample from the individual at a first time;
b) determining a first ratio of Dialister spp. to Phascolarctobacterium spp. in the stool sample;
c) providing a second stool sample from the individual at a second time, the second time being after the first time;
d) determining a second ratio of Dialister spp. To Phascolarctobacterium spp. in the second stool sample; and
e) comparing the first ratio and the second ratio, an increase of the ratio being indicative of a worsening of IBD and/or a decrease of the ratio being indicative of an improvement or regression of IBD.

The ratio of Dialister spp. to Phascolarctobacterium spp. can also be useful for treatment response monitoring purposes. Indeed, the ratio can be a useful biomarker for assessing the effectivity of a treatment of a disease or disorder associated with an intestinal dysbiosis, preferably IBD. A decreased ratio would be indicative of an effective treatment or a response to the treatment whereas an unchanged or increased ratio would be indicative of an ineffective treatment or an absence of response to the treatment.

Accordingly, the present invention relates to a method for monitoring treatment response in an individual suffering of a disease or disorder associated with an intestinal dysbiosis, comprising
a) providing a first biological sample from the individual before the beginning of a treatment or at a first time during the treatment;
b) determining a first ratio of Dialister spp. to Phascolarctobacterium spp. in the first biological sample;
c) providing a second biological sample from the individual at a second time during the treatment or at the end of the treatment, the second time being after said beginning and said first time;
d) determining a second ratio of Dialister spp. to Phascolarctobacterium spp. in the second biological sample; and
e) comparing the first ratio and the second ratio, a decrease of the ratio being indicative of an effective treatment of the disease or disorder associated with an intestinal dysbiosis in the individual or a response to the treatment and/or an unchanged ratio or an increase of the ratio being indicative of an ineffective treatment or an absence of response to the treatment.

Alternatively, the present invention relates to a method for monitoring treatment response in a subject suffering of a disease or disorder associated with an intestinal dysbiosis, comprising:
a) providing a first biological sample from the subject at a first time;
b) determining a first ratio of Phascolarctobacterium spp. to Dialister spp. in the first biological sample;
c) providing a second biological sample from the subject at a second time, the second time being after the first time;
d) determining a second ratio of Phascolarctobacterium spp. to Dialister spp. in the second biological sample; and
e) comparing the first ratio and the second ratio, an increase of the ratio being indicative of an effective treatment of said disease or disorder associated with an intestinal dysbiosis in the subject.

Preferably, the biological sample is a stool sample.

Preferably, the disease or disorder associated with an intestinal dysbiosis is selected from the group consisting of IBD, particularly ulcerative colitis or Crohn's disease, and low body mass index (underweight), preferably is IBD.

In particular, the present invention relates to a method for monitoring treatment response in an individual suffering of IBD, comprising
a) providing a first stool sample from the individual before the beginning of a treatment or at a first time during the treatment;
b) determining a first ratio of Dialister spp. to Phascolarctobacterium spp. in the first stool sample;
c) providing a second stool sample from the individual at a second time during the treatment or at the end of the treatment, the second time being after said beginning and said first time;
d) determining a second ratio of Dialister spp. to Phascolarctobacterium spp. in the second stool sample; and
e) comparing the first ratio and the second ratio, a decrease of the ratio being indicative of an effective treatment of IBD in the individual or a response to the treatment and/or an unchanged ratio or an increase of the ratio being indicative of an ineffective treatment or an absence of response to the treatment.

Optionally, the ratio can be determined at the beginning of the treatment and at the end of the treatment. Optionally, the ratio can be determined before the administration of the treatment and after the administration of the treatment. Optionally, the ratio can be determined at a first time during the treatment and at a second time during the treatment. Optionally, the ratio can be determined at the beginning of the treatment and at a time during the treatment.

Optionally, the treatment is a dietary intervention, a treatment with a drug or a medicament candidate, or a probiotic administration. The term “drug” includes synthesized pharmaceuticals but also biopharmaceuticals or biological medical products, such as vaccines, blood components, somatic cells, gene therapies, tissues, recombinant therapeutic proteins, etc. In particular, the term “drug” includes living medicines and preferably formulations comprising bacterial cells selected or engineered to possess therapeutic properties.

In some embodiments, the treatment is a probiotic composition comprising or consisting of Phascolarctobacterium spp., preferably Phascolarctobacterium faecium and/or Phascolarctobacterium_A succinatutens, and optionally Prevotella spp. Preferably, such probiotic does not comprise Dialister spp.

The ratio of Dialister spp. to Phascolarctobacterium spp. can also be useful for stratifying patient population in a clinical trial, in particular a clinical trial including subjects having a disease or disorder associated with an intestinal dysbiosis, preferably IBD, in particular clinical trials of candidate medicament for the treatment of said disease or disorder. Indeed, depending on the ratio Dialister spp. to Phascolarctobacterium spp, the patients of the clinical trial may have a different response to the candidate medicament. This information could thus be useful for selecting the most appropriate patient population for a given candidate medicament.

The ratio of Dialister spp. to Phascolarctobacterium spp. can also be useful for stratifying patient population in a clinical trial, in particular a clinical trial including IBD subjects, optionally subjects suffering of ulcerative colitis and Crohn's disease, in particular clinical trials of candidate medicament for the treatment of IBD.

The presence or absence of Prevotella spp. can further be useful for stratifying patient population in a clinical trial, in particular a clinical trial including subjects having a disease or disorder associated with an intestinal dysbiosis in particular IBD.

The ratio of Dialister spp. to Phascolarctobacterium spp. can also be useful for selecting a subject suffering from dysbiosis caused by an altered ratio between Dialister spp. and Phascolarctobacterium spp. Such subject will benefit from a treatment that will be able to shift the Dialister spp. to Phascolarctobacterium spp. ratio. In particular, subjects with D/P ratio superior to 1 or with P/D ratio inferior to 1 are subjects susceptible to benefit from a treatment as disclosed here below.

The invention thus concerns a method for selecting a subject suffering from a disease or disorder associated with an intestinal dysbiosis as susceptible to benefit from a treatment comprising the administration of Phascolarctobacterium spp., wherein the method comprises:
a) providing a biological sample from the subject;
b) determining a ratio of Dialister spp. to Phascolarctobacterium spp. (D/P) or a ratio of Phascolarctobacterium spp. to Dialister spp. (P/D) in the biological sample; and,
c) selecting the subject based on the determined ratio of Dialister spp. to Phascolarctobacterium spp. (D/P) or Phascolarctobacterium spp. to Dialister spp. (P/D) as susceptible to benefit from the treatment, in particular selecting the subject if the D/P ratio is superior to 1 or the P/D ratio is inferior to 1.

Preferably, the disease or disorder associated with intestinal dysbiosis is IBD and the method further comprises the determination of the presence or absence of Prevotella spp. in the biological sample, and the subject is susceptible to benefit from the treatment if i) the ratio D/P is superior to 1 or the P/D ratio is inferior to 1, and ii) Prevotella is absent.

Optionally, the treatment is a dietary intervention, a treatment with a drug or a medicament candidate, or a probiotic administration. The term “drug” includes synthesized pharmaceuticals but also biopharmaceuticals or biological medical products, such as vaccines, blood components, somatic cells, gene therapies, tissues, recombinant therapeutic proteins, etc. In particular, the term “drug” includes living medicines and preferably formulations comprising bacterial cells selected or engineered to possess therapeutic properties.

In some embodiments, the treatment is a probiotic composition comprising or consisting of Phascolarctobacterium spp., preferably Phascolarctobacterium faecium and/or Phascolarctobacterium_A succinatutens, and optionally Prevotella spp. Preferably, such probiotic does not comprise Dialister spp.

The invention particularly concerns a method for selecting a subject susceptible to benefit from a treatment capable of modifying a ratio of Dialister spp. To Phascolarctobacterium spp. (D/P), in particular capable of decreasing the D/P ratio below 1, comprising:
a) providing a stool sample from the subject;
b) determining a ratio of Dialister spp. to Phascolarctobacterium spp. in the stool sample;
c) selecting the subject based on the determined ratio of Dialister spp. to Phascolarctobacterium spp., as susceptible to benefit from the treatment, in particular if the ratio is higher than 1; and
d) optionally, treating the subject with a treatment capable of modifying the ratio of Dialister spp. to Phascolarctobacterium spp., so that the D/P ratio is lower than 1, preferably lower than 0.95, 0.9, 0.8 0.7, 0.6 or 0.5.

Preferably, the modification of the ratio of Dialister spp. to Phascolarctobacterium spp. allows the prevention or treatment of a disease or disorder associated with an intestinal dysbiosis, preferably the prevention or treatment of IBD.

In an embodiment, the methods disclosed herein further comprise a step of administering a treatment, in particular a veterinary or pharmaceutical composition comprising Phascolarctobacterium spp. and optionally Prevotella spp. or a combined preparation for simultaneous, separate or sequential use comprising i) an agent able to decrease Dialister spp. population in a subject and ii) an agent able to increase Phascolarctobacterium spp. population in a subject.

Finally, the ratio of Dialister spp. to Phascolarctobacterium spp. can be useful for developing new treatment of a disease or disorder associated with an intestinal dysbiosis, preferably IBD, particularly ulcerative colitis or Crohn's disease, colorectal cancer, atherosclerotic cardiovascular disease and low body mass index (underweight). The treatment can be selected for being capable of modifying the ratio of Dialister spp. to Phascolarctobacterium spp., especially for being capable of decreasing this ratio in the case of IBD and underweight or being capable of increasing this ratio in the case of colorectal cancer and atherosclerotic cardiovascular disease.

Accordingly, the present invention relates to a method of treatment of a disease or disorder associated with an intestinal dysbiosis, preferably IBD, in a subject comprising administering a therapeutically effective amount of a veterinary or pharmaceutical composition comprising an agent capable of modifying the ratio of Dialister spp. to Phascolarctobacterium spp., especially for being capable of decreasing this ratio, to said subject. In addition, the present invention relates to this agent or a veterinary or pharmaceutical composition comprising this agent for use in the treatment of a disease or disorder associated with an intestinal dysbiosis and to the use of this agent or a veterinary or pharmaceutical composition comprising this agent for the manufacture of a medicament for the treatment of a disease or disorder associated with an intestinal dysbiosis.

Preferably, the disease or disorder associated with an intestinal dysbiosis is selected from the group consisting of IBD, particularly ulcerative colitis or Crohn's disease, colorectal cancer, atherosclerotic cardiovascular disease and low body mass index (underweight), preferably is IBD.

Particularly, the present invention relates to a method of treatment of IBD in a subject comprising administering a therapeutically effective amount of a veterinary or pharmaceutical composition comprising an agent capable of modifying the ratio of Dialister spp. to Phascolarctobacterium spp., especially for being capable of decreasing this ratio, to said subject. In addition, the present invention relates to this agent or a veterinary or pharmaceutical composition comprising this agent for use in the treatment of IBD and to the use of this agent or a veterinary or pharmaceutical composition comprising this agent for the manufacture of a medicament for the treatment of IBD.

Optionally, the agent capable of modifying the ratio of Dialister spp. to Phascolarctobacterium spp., especially for being capable of decreasing this ratio, is a composition comprising Phascolarctobacterium spp. and optionally Prevotella spp. Such composition may be used after or in combination with an agent specifically targeting Dialister spp., and reducing or eliminating the population of Dialister spp. such as an antibiotic agent having an effect against Dialister spp., in particular a specific effect against Dialister spp., selective nutrition, modulation of transit time and/or cleansing of an in situ microbiome space. Non-limiting examples of antibiotic agents are antibiotics targeting anaerobic gut bacteria, preferably Firmicutes, more preferably Tenericutes for instance Vancomycin.

In a particular aspect, the subject has a ratio of Dialister spp. to Phascolarctobacterium spp. associated with a subject suffering or at risk of developing inflammatory bowel disease (IBD), in particular if the ratio is higher than 1.

Thus, in a specific aspect, the present invention relates to a method of treatment of a disease or disorder associated with an intestinal dysbiosis, preferably IBD, particularly ulcerative colitis or Crohn's disease, in a subject comprising administering a therapeutically effective amount of a veterinary or pharmaceutical composition comprising Phascolarctobacterium spp. to said subject, in particular if the D/P ratio is higher than 1 in the intestinal microbiome of said subject.

In another aspect, the subject has a ratio of Dialister spp. to Phascolarctobacterium spp. associated with a subject suffering or at risk of developing colorectal cancer or atherosclerotic cardiovascular disease, in particular if the ratio is lower than 1.

Thus, in another specific aspect, the present invention also relates to a method of treatment of a disease or disorder associated with an intestinal dysbiosis, such as CRC and ACVD, in a subject comprising administering a therapeutically effective amount of a veterinary or pharmaceutical composition comprising Dialister spp. to said subject, in particular if the D/P ratio is lower than 1 in the intestinal microbiome of the said subject.

“An effective amount” or a “therapeutic effective amount” as used herein refers to the amount of active agent required to confer therapeutic effect on the subject, either alone or in combination with one or more other active agents, e.g., the amount of active agent that is needed to treat the targeted disease or disorder, or to produce the desired effect. The “effective amount” will vary depending on the agent(s), the disease and its severity, the characteristics of the subject to be treated including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

In an aspect, the present invention relates to a method of treatment of a disease or disorder associated with an intestinal dysbiosis, preferably IBD, in a subject comprising administering a therapeutically effective amount of a veterinary or pharmaceutical composition comprising an agent capable of decreasing the ratio of Dialister spp. to Phascolarctobacterium spp. (D/P), or increasing the ratio of Phascolarctobacterium spp. to Dialister spp. (P/D) to said subject.

In a very specific aspect, the present invention relates to a method of treatment of a disease or disorder associated with an intestinal dysbiosis, preferably IBD, particularly ulcerative colitis or Crohn's disease, in a subject comprising administering a therapeutically effective amount of a veterinary or pharmaceutical composition comprising Phascolarctobacterium spp. and Prevotella spp. to said subject, in particular if the D/P ratio is higher than 1 and optionally if Prevotella spp. is absent from the intestinal microbiome of said subject.

If the disease or disorder associated with an intestinal dysbiosis is colorectal cancer or atherosclerotic cardiovascular disease, the method may comprise administering a therapeutically effective amount of a veterinary or pharmaceutical composition comprising Dialister spp. to said subject.

It also relates to a veterinary or pharmaceutical composition comprising Phascolarctobacterium spp. and optionally Prevotella spp. for use in the treatment of a disease or disorder associated with an intestinal dysbiosis, preferably IBD, particularly ulcerative colitis or Crohn's disease, and to the use of a veterinary or pharmaceutical composition comprising Phascolarctobacterium spp. and optionally Prevotella spp. for the manufacture of a medicament for the treatment of a disease or disorder associated with an intestinal dysbiosis, preferably IBD, particularly ulcerative colitis or Crohn's disease.

Such composition may be used after or in combination with an agent specifically targeting Dialister spp., and reducing or eliminating the population of Dialister spp. such as an antibiotic agent having an effect against Dialister spp., in particular a specific effect against Dialister spp., selective nutrition, modulation of transit time and/or cleansing of an in situ microbiome space.

Conversely, the veterinary or pharmaceutical composition may comprise Dialister spp. and may be for use in the treatment of a disease or disorder associated with an intestinal dysbiosis which is CRC or ACVD.

In a specific aspect, the present invention relates to a method of treatment of IBD in a subject comprising administering a therapeutically effective amount of a veterinary or pharmaceutical composition comprising Phascolarctobacterium spp. to said subject. The Phascolarctobacterium spp. administration allows the decrease of the ratio of Dialister spp. to Phascolarctobacterium spp. It also relates to a veterinary or pharmaceutical composition comprising Phascolarctobacterium spp. for use in the treatment of IBD and to the use of a veterinary or pharmaceutical composition comprising Phascolarctobacterium spp. for the manufacture of a medicament for the treatment of IBD. Such composition may be used after or in combination with an agent specifically targeting Dialister spp., and reducing or eliminating the population of Dialister spp. such as an antibiotic agent having an effect against Dialister spp., in particular a specific effect against Dialister spp., selective nutrition, modulation of transit time and/or cleansing of an in situ microbiome space. In a particular aspect, the subject has a ratio of Dialister spp. to Phascolarctobacterium spp. associated with a subject suffering or at risk of developing inflammatory bowel disease (IBD), in particular a ratio higher than 1.

In a preferred embodiment, the method of treatment of a disease or disorder associated with an intestinal dysbiosis, preferably IBD, particularly ulcerative colitis or Crohn's disease, in a subject comprises a step of reducing or eliminating the population of Dialister spp. in the subject, preferably by pre-treatment with antibiotics, selective nutrition, modulation of transit time and/or cleansing of an in situ microbiome space.

In a preferred aspect, the method of treatment of a disease or disorder associated with an intestinal dysbiosis, preferably IBD, particularly ulcerative colitis or Crohn's disease, in a subject comprising administering a therapeutically effective amount of a veterinary or pharmaceutical composition comprising an agent capable of modifying the ratio of Dialister spp. to Phascolarctobacterium spp., to said subject includes a step of:
reducing or eliminating the population of Dialister spp. in the subject, preferably by a pre-treatment such as antibiotic pre-treatment, selective nutrition, modulation of transit time and/or rinsing or cleansing of an in situ microbiome space, treatment by means of phages and/or substrate scavenging agents, prior to administering the therapeutically effective amount of a veterinary or pharmaceutical composition comprising Phascolarctobacterium spp. to said subject.

Particularly preferred is the treatment by means of phages. Methods to identify suitable phages are known to one of skill in the art (e.g., Rands, C. M., Brüssow, H. and Zdobnov, E. M. (2019), Comparative genomics groups phages of Negativicutes and classical Firmicutes despite different Gram-staining properties. Environ Microbiol, 21:3989-4001).

It is an advantage of said pretreatment that the previous reduction of Dialister spp. population combined with a subsequent administration of a composition comprising Phascolarctobacterium spp. provides to Phascolarctobacterium spp. a competitive advantage over Dialister spp. and allows purposefully shift in favor of Phascolarctobacterium spp.

Suitable antibiotics include antibiotics targeting anaerobic gut bacteria, preferably Firmicutes, more preferably Tenericutes for instance Vancomycin. Selective nutrition includes the elimination of specific nutrients (i.e., fibers, proteins). The transit time may for example be modified by using laxatives. Cleansing might be achieved by administration of laxatives, salts and/or similar. It is an advantage of said pre-treatment that the previous reduction of Dialister spp. population combined with a subsequent administration of a designed composition allows Phascolarctobacterium spp. a competitive advantage over Dialister spp. such that the microbiome's population is shifted in favor of Phascolarctobacterium spp.”

In a specific aspect, the invention relates to a method of prophylactic treatment of in a subject, comprising the steps for assessing whether an individual is at risk of developing IBD as described above and additionally including the step of administering a therapeutically effective amount of a veterinary or pharmaceutical composition comprising an agent capable of modifying the ratio of Dialister spp. to Phascolarctobacterium spp. to the subject, preferably a composition comprising Phascolarctobacterium spp, optionally in combination with an agent able to decrease Dialister spp. Population such as an antibiotic agent, selective nutrition, modulation of transit time and/or cleansing of an in situ microbiome space.

This method makes use of the knowledge about risks of developing IBD, which is obtainable due to the assessment method as described above and includes a step for preventing materialization of the said risks. The invention also relates to a veterinary or pharmaceutical composition comprising Phascolarctobacterium spp. for use in the prophylactic treatment of a patient being identified with a ratio of Dialister spp. to Phascolarctobacterium spp. associated with a subject at risk of developing a disease or disorder associated with an intestinal dysbiosis, preferably IBD, particularly ulcerative colitis or Crohn's disease, in particular higher than 1 according to the method as described above.

Particularly, the invention concerns a veterinary or pharmaceutical composition comprising Phascolarctobacterium spp. for use in the treatment of a subject having a disease or disorder associated with an intestinal dysbiosis, said subject having a Dialister spp. to Phascolarctobacterium spp. (D/P) ratio superior to 1 or a Phascolarctobacterium spp. to Dialister spp. (P/D) ratio inferior to 1.

Alternatively, the invention relates to a veterinary of pharmaceutical composition comprising Dialister spp. for use in the prophylactic treatment of a patient being identified with a ratio of Dialister spp. to Phascolarctobacterium spp. associated with a subject at risk of developing a disease or disorder associated with an intestinal dysbiosis such as CRC and ACVD.

In a preferred aspect, the veterinary or pharmaceutical composition comprising Phascolarctobacterium spp. for use in the treatment of a disease or disorder associated with an intestinal dysbiosis, preferably IBD, particularly ulcerative colitis or Crohn's disease, and/or for use in the prophylactic treatment of a patient at risk of developing a disease or disorder associated with an intestinal dysbiosis, preferably IBD, particularly ulcerative colitis or Crohn's disease, as described above is a composition comprising a consortium of viable live bacteria strains, in particular which have been obtained in a co-cultivation process. Preferably the composition also comprises intermediate metabolites, end metabolites and dispersing medium. It is particularly preferred that the composition is a composition as described in WO2018/189284 and/or WO2020/079026. The teachings of these documents are included herein by reference.

Preferably, the veterinary or pharmaceutical composition comprises Phascolarctobacterium spp., preferably viable Phascolarctobacterium spp., in particular viable Phascolarctobacterium faecium and/or Phascolarctobacterium_A succinatutens bacteria. Such composition preferably does not comprise bacteria from the Dialister genera.

In some embodiments, the veterinary or pharmaceutical composition further comprises Prevotella spp., preferably viable Prevotella spp., particularly Prevotella copri (DSM 18205, JCM 13464) or Prevotella stercorea (DSM 18206, JCM 13469), Prevotella oris JCM 12252, Prevotella hominis.

Alternatively, the veterinary or pharmaceutical composition comprises Dialister spp., preferably Dialister hominis (JCM 33369, DSM 109768), Dialister invisus (DSM 15470, JCM 17566), Dialister propionifaciens (JCM 17568), Dialister succinatiphilus (DSM 21274, JCM 15077). Such composition is particularly useful for the treatment of diseases or disorders in which the D/P ratio is inferior to 1, such as CRC or AVSD. Such composition preferably does not comprise Phascolarctobacterium.

The terms “viable bacteria” and/or “live bacteria” are known in the field; in particular, they denote bacteria, wherein viable bacterial strains have the capacity to grow under suitable conditions and live bacterial indicate viability as measured using biochemical assays. The term viable, live bacterial strains in particular relates to bacterial strains (i) having a viability of over 50% (e.g., in pharmaceutical products), typically over 60% such as over 90% (e.g., in products manufactured according to the inventive method) as determined by flow cytometry. Viability over 90% is typically observed in the compositions as initially obtained by continuous cultivation and by batch or fed-batch cultivation, viability over 60% is typically observed after stabilization.

The term “consortium”, “microbial consortium” or “bacterial consortium” refers herein to at least two or at least three microbial organisms, preferably officiating in the same metabolic or trophic network. Preferably, microbial members of the consortium collaborate, in particular for their subsistence, into the consortium. More preferably, each bacterium of the consortium (i) produces a compound which is utilized by another bacterium of the consortium and/or (ii) utilizes a compound which is produced by another bacterium of the consortium.

It is particularly advantageous when the veterinary or pharmaceutical composition comprising Phascolarctobacterium spp. is an in vitro assembled consortium of selected live, viable bacterial strains obtained by anaerobic co-cultivation in a dispersing medium, wherein the consortium comprises
at least one bacterial strain consuming succinate and producing propionate and acetate, wherein this bacterial strain pertains to Phascolarctobacterium spp.;
and at least one bacterial strain consuming sugars, fibers, and resistant starch, and producing succinate, preferably a bacterial strain selected from the genera Alistipes, Bacteroides, Blautia, Barnesiella, Clostridium, Ruminococcus and Prevotella, more preferably Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Ruminococcus callidus, Ruminococcus flavefaciens, Prevotella copri, Prevotella stercorea, Alistipes finegoldii, Alistipes onderdonkii, Alistipes shahii and any combination thereof.

It is particularly preferred that the composition comprising Phascolarctobacterium spp. is a composition comprising viable, live bacteria strains (i), intermediate metabolites, (ii) end metabolites (iii) and a dispersing medium (iv), characterized in that said bacteria strains (i) being selected from:
(A1) strains consuming sugars, fibers, and resistant starch, producing formate and acetate, and being selected from the genera Ruminococcus, Dorea and Eubacterium;
(A2) strains consuming sugars, starch and acetate, producing formate and butyrate, and being selected from the genera Faecalibacterium, Roseburia and Anaerostipes;
(A3) strains consuming sugars and oxygen, producing lactate, and being selected from the genera Lactobacillus, Streptococcus, Escherichia, Lactococcus and Enterococcus;
(A4) strains consuming sugars, starch, and carbon dioxide, producing lactate, formate and acetate, and being selected from the genus Bifidobacterium;
(A5) strains consuming lactate or proteins, producing propionate and acetate, and being selected from the genera Clostridium, Propionibacterium, Veillonella and Megasphaera;
(A6) strains consuming lactate and starch, producing acetate, butyrate and hydrogen, and being selected from the genera Eubacterium, Clostridium and Anaerostipes;
(A7) strains consuming sugar, starch, formate producing lactate, formate and acetate, and being selected from the genus Collinsella;
(A8) strains consuming succinate, producing propionate and acetate, and being selected from the genera Phascolarctobacterium; and
(A9) strains consuming sugars, fibers, formate and hydrogen, producing acetate and butyrate and being selected from the genera Acetobacterium, Blautia, Clostridium, Moorella, Methanobrevibacter, Methanomassiliicoccus and Sporomusa; and
(A10) strains consuming sugars, fibers, and resistant starch, and producing succinate, preferably a bacterial strain selected from the genera Alistipes, Bacteroides, Blautia, Barnesiella, Clostridium, Ruminococcus and Prevotella;
wherein said bacteria strains are in each case identified through classification of the full 16S gene with assignment for the different taxonomic levels Phylum: 75%, Class: 78.5%, Order: 82%, Family: 86.5%, Genus: 94.5%, sequence similarity;
wherein preferably bacteria strains (i) of all ten groups (A1) to (A10) are present;
wherein at least one bacteria strain pertains to Phascolarctobacterium spp. and at least bacteria strain is a representative of A10;
said intermediate metabolites (ii) being selected from:
   succinate in an amount of less than 5 mM, and
   formate in an amount of less than 5 mM, and
   lactate in an amount of less than 5 mM; and
said end metabolites (iii) being selected from
   acetate in an amount of at least 10 mM, and
   propionate in an amount of at least 2 mM, and
   butyrate in an amount of at least 2 mM; and
said dispersing medium (iv) preferably being selected from:
   culture media, or
   cryoprotecting media, or
   aqueous gels, or
   polymeric supports.

In one embodiment, the veterinary or pharmaceutical composition comprises:
at least one bacterial strain selected from the genera Ruminococcus, Dorea, Clostridium and Eubacterium;
at least one bacterial strain selected from the genera Faecalibacterium, Roseburia, Anaerostipes and Eubacterium;
at least one bacterial strain selected from the genera Lactobacillus, Streptococcus, Escherichia, Lactococcus and Enterococcus;
at least one bacterial strain of the genus Bifidobacterium or Roseburia;
at least one bacterial strain selected from the genera Clostridium, Propionibacterium, Veillonella,
Coprococcus and Megasphaera;
at least one bacterial strain selected from the genera Anaerostipes, Clostridium and Eubacterium (A6);
at least one bacterial strain of the genus Collinsella or Roseburia;
at least one bacterial strain selected from the genera Phascolarctobacterium; and
at least one bacterial strain selected from the genera Blautia, Eubacterium and an archaea of the genus Methanobrevibacter or Methanomassiliicoccus;
optionally at least one bacterial strain selected from the genera Alistipes, Bacteroides, Blautia, Clostridium, Ruminococcus and Prevotella; and
optionally
at least one bacterial strain selected from the genera Alistipes, Bacteroides, Blautia, Barnesiella, Clostridium, Ruminococcus and Prevotella, optionally selected from the genera Alistipes, Bacteroides, Blautia, Clostridium, Ruminococcus and Prevotella, preferably Alistipes, Bacteroides, Barnesiella, Ruminococcus and Prevotella;
at least one bacterial strain selected from the genera Clostridium, Coprococcus, Eubacterium, Flavonifractor and Flintibacter;
at least one bacterial strain selected from the genera Bacteroides, Barnesiella, Bifidobacterium, Clostridium (only tryptamine producers), Enterococcus, Faecalibacterium, Lactobacillus and Ruminococcus (only tryptamine producers);
at least one bacterial strain selected from the genera Anaerostipes, Blautia, Clostridium and Faecalibacterium;
at least one bacterial strain selected from the genera Bacteroides, Bifidobacterium, Blautia, Clostridium, Faecalibacterium, Lactobacillus, Prevotella and Ruminococcus; and/or
at least one bacterial strain selected from the genera Akkermansia, Bacteroides, Bifidobacterium and Ruminococcus.

In an aspect, the veterinary or pharmaceutical composition according to the invention comprises: Ruminococcus bromii, Faecalibacterium prausnitzii, Lactobacillus rhamnosus, Bifidobacterium adolescentis, Anaerotignum lactatifermentans, Eubacterium limosum, Collinsella aerofaciens, Phascolarctobacterium faecium, and Blautia hydrogenotrophica and optionally Bacteroides xylanisolvens.

In another aspect, the veterinary or pharmaceutical composition according to the invention comprises: Ruminococcus bromii, Faecalibacterium prausnitzii, Lactobacillus rhamnosus, Bifidobacterium adolescentis, Anaerotignum lactatifermentans, Eubacterium limosum, Collinsella aerofaciens and Phascolarctobacterium faecium and optionally Bacteroides xylanisolvens.

In a preferred embodiment, the composition comprising Phascolarctobacterium spp. or Dialister spp respectively is a composition comprising a consortium of live bacterial strains, wherein the consortium may comprise:
at least one bacterial strain being able to perform metabolic pathway p10, p10 corresponding to the conversion of succinate and the production of propionate, and at least one bacterial strain being able to perform metabolic pathway p5, p5 corresponding to the conversion of primary substrates, such as sugars, starches, fibers and/or proteins, and to the production of succinate;
optionally: in combination with at least one bacterial strain being able to perform metabolic pathway p6, p6 corresponding to the conversion of formate and the production of acetate, and at least one bacterial strain being able to perform metabolic pathway p1, p1 corresponding to the conversion of primary substrates and the production of formate; and
optionally: in combination with at least one bacterial strain being able to perform metabolic pathway p8 or p9, p8 corresponding to the conversion of lactate and the production of butyrate and p8 corresponding to the conversion of lactate and the production of propionate, and at least one bacterial strain being able to perform metabolic pathway p4, p4 corresponding to the conversion of primary substrates to and the production of lactate.

Experiments are known to test if a bacterial strain is able to perform a metabolic pathway and thus belongs to a particular functional group. For example, the degradation of sugars, starches or fibers can be tested simply by providing such substrate to bacteria while observing or monitoring their growth. For example, bacteria can be characterized for growth and metabolite production on MZGSC Medium (ATCC Medium 2857) and modifications thereof whereby the carbon sources glucose, cellobiose and starch are replaced by specific substrates including intermediate metabolites and/or fibers, preferably such as found in the human intestine. The concentrations of the produced metabolites can for example be quantified by any analytic method available for the person skilled in the art such as refractive index detection high pressure liquid chromatography (HPLC-RI; for example, as provided by Thermo Scientific Accela).

The bacterial strains being able to perform metabolic pathway p10 may be selected from the genera Dialister, Flavonifractor, Phascolarctobacterium and Veillonella. For example, the bacterial strains may be selected from the species Dialister hominis (JCM 33369, DSM 109768), Dialister invisus (DSM 15470, JCM 17566), Dialister propionifaciens (JCM 17568), Dialister succinatiphilus (DSM 21274, JCM 15077), Flavonifractor plautii (ATCC 29863,DSM 4000), Phascolarctobacterium faecium (DSM 14760), Phascolarctobacterium succinatutens (DSM 22533, JCM 16074), Veillonella parvula (ATCC 10790, DSM 2008, JCM 12972), Veillonella ratti (ATCC 17746, DSM 20736, JCM 6512). Preferably, a bacterial strain being able to perform metabolic pathway p10 is Phascolarctobacterium faecium (DSM 14760) or Phascolarctobacterium succinatutens (DSM 22533, JCM 16074).

The bacterial strains being able to perform metabolic pathway p5 may be selected from the genera Bacteroides, Phocaeicola, Prevotella, Alistipes, Bacteroides, Blautia, Clostridium, Dorea and Ruminococcus. For example, the bacterial strains may be selected from the species Alistipes finegoldii (DSM 1724, JCM 16770), Alistipes onderdonkii (ATCC BAA-1178, DSM19147, JCM 16771), Alistipes shahii (ATCC BAA-1179, DSM 19121, JCM 16773), Bacteroides caecimuris, Bacteroides faecis (DSM 24798, JCM 16478), Bacteroides fragilis (ATCC 25285, DSM 2151, JCM 11019), Bacteroides ovatus (ATCC 8483, DSM 1896, JCM 5824), Bacteroides plebeius (DSM 17135, JCM 12973), Bacteroides thetaiotaomicron (ATCC 29148, DSM 2079, JCM 5827), Bacteroides uniformis (ATCC 8492, DSM 6597, JCM5828), Bacteroides vulgatus (ATCC8482, DSM 1447, JCM 5826), Bacteroides xylanisolvens (DSM 18836, JCM 15633), Blautia luti (DSM 14534, JCM 17040), Blautia wexlerae (ATCC BAA-1564, DSM 19850, JCM 17041), Blautia/Clostridium coccoides (ATCC 29236, DSM 935, JCM 1395), Clostridium bartlettii (ATCC BAA-827, DSM 16795), Clostridium butyricum (ATCC 19398, DSM 10702, JCM 1391), Phocaeicola dorei, Phocaeicola plebeius, Phocaeicola vulgatus, Prevotella copri (DSM 18205, JCM 13464), Prevotella stercorea (DSM 18206, JCM 13469), Ruminococcus callidus (ATCC 27760), Ruminococcus flavefaciens (DSM 25089). Preferably, the bacterial strain being able to perform metabolic pathway p5 is Bacteroides faecis (DSM 24798, JCM 16478), Bacteroides fragilis (ATCC 25285, DSM 2151, JCM 11019), Bacteroides thetaiotaomicron (ATCC 29148, DSM 2079, JCM 5827), Bacteroides uniformis (ATCC 8492, DSM 6597, JCM5828), Bacteroides xylanisolvens (DSM 18836, JCM 15633), Prevotella copri (DSM 18205, JCM 13464) or Prevotella stercorea (DSM 18206, JCM 13469).

The bacterial strains being able to perform metabolic pathway p6 may be selected from the genera Acetobacterium, Blautia, Candidatus Methanomassiliicoccus, Clostridium, Eubacterium, Methanobrevibacter and Veillonella. For example, the bacterial strains may be selected from the species Acetobacterium carbinolicum (ATCC BAA-990, DSM 2925), Acetobacterium malicum (DSM 4132), Acetobacterium wieringae (ATCC 43740, DSM1911, JCM 2380), Blautia hydrogenotrophica (DSM 10507, JCM 14656), Blautia producta (ATCC 27340, DSM 2950, JCM 1471), Candidatus Methanomassiliicoccus intestinalis, Clostridium aceticum (ATCC 35044, DSM 1496, JCM 15732), Clostridium glycolicum ((ATCC 14880, DSM 1288, JCM 1401), Clostridium magnum (ATCC 49199, DSM 2767), Clostridium mayombe (ATCC 51428, DSM 2767), Eubacterium callanderi (ATCC 49165, DSM 3662, JCM 10284), Eubacterium hallii (ATCC 27751, DSM 3353, JCM 31263), Eubacterium limosum (ATCC 8486, DSM 20543, JCM 6421), Eubacterium ramulus (A TCC 29099, DSM 15684, JCM), Methanobrevibacter smithii (ATCC 35061, DSM 861, JCM 328), Veillonella parvula (ATCC 10790, DSM 2008, JCM 12972), Veillonella ratti (ATCC 17746, DSM 20736, JCM 6512). Preferably, the bacterial strain being able to perform metabolic pathway p6 is Blautia hydrogenotrophica (DSM 10507, JCM 14656), Blautia producta (ATCC 27340, DSM 2950, JCM 1471) or Eubacterium callanderi (ATCC 49165, DSM 3662, JCM 10284).

The bacterial strains being able to perform metabolic pathway p1 may be selected from the genera Agathobacter, Anaerobutyricum, Anaerostipes, Bacteroides, Bariatrics, Bifidobacterium, Blauta, Clostridium, Collinsella, Coprococcus, Dorea, Enterococcus, Erysipelatoclostridium, Escherichia, Eubacterium, Faecalibacterium, Lachnospira, Lacticaseibacillus, Longicatena, Phocaeicola, Prevotella, Roseburia, Ruminococcus, Schaedlerella, Sellimonas, Sutterella and Turicimona. For example, the bacterial strains may be selected from the species Agathobacter/Roseburia faecis (DSM 16840), Agathobacter rectalis/Eubacterium rectale (DSM 17629, ATCC 33656), Anaerobutyricum hallii (ATCC 27751, DSM 3353, JCM 31263), Anaerostipes caccae (DSM14662, JCM 13470), Anaerostipes hadrus (ATCC 29173, DSM 3319), Bacteroides faecis (DSM 24798, JCM 16478), Bacteroides uniformis (DSM 6597, ATCC 8492, JCM5828), Bacteroides xylanisolvens (DSM 18836, JCM 15633), Bariatricus comes, Bifidobacterium adolescentis (ATCC 15703, DSM 20083, JCM 1251), Bifidobacterium longum (ATCC 15707, DSM 20219, JCM1217), Bifidobacterium pseudocatenulatum (ATCC 27919, DSM 20438, JCM 1200), Blautia hydrogenotrophica (DSM 10507, JCM 14656), Clostridium clostridioforme/Enterocloster clostridioformis (ATCC 25537, DSM 933, JCM 1291), Clostridium innocuum (ATCC 14501, DSM 1286, JCM 1292), Clostridium scindens (DSM 5676, ATCC 35704), Collinsella aerofaciens (ATCC 25986, DSM 3979, JCM 10188), Coprococcus eutactus (ATCC 27759), Dorea formicigenerans (ATCC 27755, DSM 3992, JCM 31256), Dorea longicatena (DSM 13814, JCM 11232), Enterococcus faecalis (ATCC 29212, DSM 2570), Enterococcus faecium (ATCC 19434, DSM 20477, JCM 5804), Erysipelatoclostridium ramosum (ATCC 25582, DSM 1402, JCM 1298), Escherichia coli (ATCC 11775, DSM30083, JCM 1649), Eubacterium eligens (ATCC 27750, DSM 3376), Eubacterium ramulus (ATCC 29099, DSM 15684, JCM 31355), Faecalibacterium prausnitzii (DSM 17677, ATCC 27768, ATCC 27766, JCM 31915), Lachnospira eligens (ATCC 27750, DSM 3376), Lacticaseibacillus rhamnosus/Lactobacillus rhamnosus ((ATCC 7469, DSM 20021, JCM1136), Phocaeicola vulgatus (ATCC 8482, DSM 1447, JCM 5826), Prevotella copri (DSM 18205, JCM 13464), Roseburia intestinalis (DSM 14610, CIP 107878, JCM 31262), Roseburia intestinalis (DSM 14610, CIP 107878, JCM 31262), Ruminococcus bromii (ATCC 27255, ATCC 51896), Ruminococcus callidus (ATCC 27760), Ruminococcus champanellensis (DSM 18848, JCM 17042), Ruminococcus gnavus (ATCC 29149, ATCC 35913, JCM 6515), Ruminococcus lactaris (ATCC 29176), Ruminococcus obeum (ATCC 29174, DSM 25238, JCM 31340), Sellimonas intestinalis (JCM 30749), Sutterella wadsworthensis (ATCC 51579, DSM 14016), Turicimonas muris (DSM 26109). Preferably, the bacterial strain being able to perform metabolic pathway p1 is Ruminococcus bromii (ATCC 27255, ATCC 51896), Bifidobacterium adolescentis (ATCC 15703, DSM 20083, JCM 1251), Bacteroides faecis (DSM 24798, JCM 16478), Eubacterium ramulus (ATCC 29099, DSM 15684, JCM 31355), Faecalibacterium prausnitzii (DSM 17677, ATCC 27768, ATCC 27766, JCM 31915), Coprococcus eutactus (ATCC 27759), Anaerobutyricum hallii (ATCC 27751, DSM 3353, JCM 31263), Agathobacter rectalis/Eubacterium rectale (DSM 17629, ATCC 33656) or Dorea longicatena (DSM 13814, JCM 11232).

The bacterial strains being able to perform metabolic pathway p8 may be selected from the genera Anaerobutyricum, Anaerostipes, Clostridium and Eubacterium. For example, the bacterial strains may be selected from the species Anaerobutyricum/Eubacterium hallii (ATCC 27751, DSM 3353, JCM 31263), Anaerostipes caccae (DSM 14662, JCM 13470), Eubacterium callanderi, Clostridium indolis (ATCC 25771, DSM 755, JCM 1380), Eubacterium limosum (ATCC 8486, DSM 20543, JCM 6421), Eubacterium ramulus (ATCC 29099, DSM 15684, JCM 31355). Preferably, the bacterial strain being able to perform metabolic pathway p8 is Anaerobutyricum/Eubacterium hallii (ATCC 27751, DSM 3353, JCM 31263), Anaerostipes caccae (DSM 14662, JCM 13470) or Eubacterium callanderi ATCC 49165, DSM 3662, JCM 10284).

The bacterial strains being able to perform metabolic pathway p9 may be selected from the genera Anaerotignum, Clostridium, Coprococcus, Frisingococcus, Lachnospiracea, Megasphaera, Veillonella. For example, the bacterial strains may be selected from the species Clostridium (Anaerotignum) lactatifermentans (DSM 14214), Clostridium aminovalericum (ATCC 13725, DSM 1283, JCM 1421), Clostridium celatum (ATCC 27791, DSM 1785, JCM 1394), Clostridium neopropionicum (DSM 3847), Clostridium propionicum (ATCC 25522, DSM 1682, JCM 1430), Clostridium_E sporosphaeroides, Coprococcus_A catus (ATCC 27761), Megasphaera elsdenii (ATCC 25940, DSM 20460, JCM 1772), Veillonella atypica (ATCC 17744, DSM 20739), Veillonella montpellierensis (DSM 17217), Veillonella parvula (ATCC 17745, ATCC 10790, DSM 2008, JCM 12972), Veillonella ratti (ATCC 17746, DSM 20736, JCM 6512).

The bacterial strains being able to perform metabolic pathway p4 may be selected from the genera Agathobacter, Bacteroides, Bariatricus, Bifidobacterium, Clostridium, Collinsella, Enterococcus, Escherichia, Lachnospiraceae, Lacticaseibacillus/Lactobacillus, Lactococcus, Longicatena, Peptostreptococcus, Roseburia, Streptococcus, Sutterella. For Example, the bacterial strains may be selected from the species Agathobacter/Roseburia faecis (DSM 16840), Agathobacter rectalis/Eubacterium rectale (DSM 17629), Bacteroides faecis (DSM 24798, JCM 16478), Bacteroides uniformis (DSM 6597, ATCC 8492, JCM5828), Bariatricus comes, Bifidobacterium adolescentis (ATCC 15703, DSM 20083, JCM 1251), Bifidobacterium catenulatum (ATCC 27539, DSM 20103, JCM 1194), Bifidobacterium pseudocatenulatum (ATCC 27919, DSM 20438, JCM 1200), Collinsella aerofaciens (ATCC 25986, DSM 3979, JCM 10188), Collinsella intestinalis ((DSM 13280, JCM 10643), Collinsella stercoris (DSM 13279, JCM 10641), Enterococcus caccae (ATCC BAA-1240, DSM 19114), Enterococcus faecalis (ATCC 29212, DSM 2570), Enterococcus faecium (ATCC 19434, DSM 20477, JCM 5804), Enterococcus gallinarum (ATCC 49573, JCM 8728, DSM 24841), Escherichia coli (ATCC 11775, DSM 30083, JCM 1649), Lacticaseibacillus rhamnosus/Lactobacillus rhamnosus (ATCC 7469, DSM 20021, JCM1136), Lactococcus lactis (ATCC 19435, DSM 20481), Peptostreptococcus stomatis (DSM 17678, JCM 15636), Roseburia hominis (DSM 16839), Streptococcus anginosus (ATCC 33397, DSM 20563, JCM 12993), Streptococcus salivarius (ATCC 7073, DSM 20560, JCM 5707) or Sutterella wadsworthensis (ATCC 51579, DSM 14016). Preferably, the bacterial strain being able to perform metabolic pathway p4 is Agathobacter rectalis/Eubacterium rectale (DSM 17629), Bacteroides faecis (DSM 24798, JCM 16478), Bacteroides uniformis (DSM 6597, ATCC 8492, JCM5828), Bifidobacterium adolescentis (ATCC 15703, DSM 20083, JCM 1251) Bifidobacterium pseudocatenulatum (ATCC 27919, DSM 20438, JCM 1200), Collinsella aerofaciens (ATCC 25986, DSM 3979, JCM 10188), Enterococcus faecalis (ATCC 29212, DSM 2570) or Lacticaseibacillus rhamnosus/Lactobacillus rhamnosus (ATCC 7469, DSM 20021, JCM1136).

A bacterial strain can be able to perform a plurality of metabolic pathways p10, p5, p6, p1, p8 and/or p9, and p4 as defined above. However, it is preferred that each functional group is represented by a different bacterial strain. Such a consortium may comprise at least 6 different strains.

Additionally or alternatively to the bacterial strains able to perform one or more of the above listed pathways, the consortium may comprise bacterial strains being able to perform one or more of the following pathways:
pathway 2 (p2) corresponds to the conversion of sugars, starches, fibers or proteins and to the production of acetate;
pathway 3 (p3) corresponds to the conversion of sugars, starches, fibers or proteins to the production of butyrate;
pathway 7 (p7) corresponds to the conversion of acetate and to the production of butyrate;
pathway 11 (p11) corresponds to the conversion of sugars, starches, fibers or proteins, to the reduction of oxygen and to the production of lactate;
pathway 12 (p12) corresponds to the conversion of hydrogen, carbon dioxide or formate and to the production of acetate;
pathway 13 (P13) corresponds to the conversion of peptides and to the production of propionate.

These pathways may or may not be performed by bacterial strains different from the set of strains fulfilling functions p10, p5, p6, p1, p8 and/or p9. Exemplary species can be found in WO2020/079026. Such a consortium may comprise at least 6,7,8,9,10,11 or 12 different bacterial strains.

Preferably, the veterinary or pharmaceutical composition according to the invention does not comprise bacteria from Dialister genera.

Particularly, the veterinary or pharmaceutical composition according to the invention comprises fresh, lyophilized or frozen bacteria. Preferably, the veterinary or pharmaceutical composition according to the invention comprises viable or living bacteria.

It is an advantage of such a composition comprising viable, live bacteria strains that it is designed such that Phascolarctobacterium spp. is strengthened when competing with (potentially) residual Dialister spp. for succinate as a substrate.

Thus, in another aspect, the invention relates to a veterinary or pharmaceutical composition such as described above for use in the treatment of a subject having a Dialister spp. to Phascolarctobacterium spp. ratio associated with a subject at risk of developing inflammatory bowel disease (IBD), in particular higher than 1, in particular higher to 1.05, 1.1, 1.2, 1.3, 1.4 or 1.5, and optionally suffering from IBD. The invention relates to the use of a veterinary or pharmaceutical composition such as described above for the manufacture of a medicament for use for treating a subject having a Dialister spp. to Phascolarctobacterium spp. ratio associated with a subject at risk of developing inflammatory bowel disease (IBD), in particular higher than 1, in particular higher to 1.05, 1.1, 1.2, 1.3, 1.4 or 1.5, and optionally suffering from IBD. It also relates to a method for treating a subject having a Dialister spp. to Phascolarctobacterium spp. ratio associated with a subject at risk of developing inflammatory bowel disease (IBD), in particular higher than 1, in particular higher to 1.05, 1.1, 1.2, 1.3, 1.4 or 1.5, and optionally suffering from IBD, comprising administering a therapeutically effective amount of a veterinary or pharmaceutical composition such as described above.

The invention may also concern a “combined preparation”, or a “kit of parts” in the sense that the combination partners (a) and (b) can be administered independently or by use of different fixed combinations with distinguished amounts of the combination partners, i.e., simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can vary. The combination partners can be administered by the same route or by different routes. Such combined preparation comprises a) an agent able to decrease Dialister spp. population and b) an agent able to increase Phascolarctobacterium spp. population.

The agent able to decrease Dialister spp. population is to be administered sequentially or simultaneously with the agent able to increase Phascolarctobacterium spp. population.

In an embodiment, the kit of parts comprises i) an agent able to decrease Dialister spp. population in a subject, ii) an agent able to increase Phascolarctobacterium spp. population in a subject and optionally iii) an agent able to increase Prevotella spp., as a combined preparation for simultaneous, separate or sequential use, for use in the treatment of a subject having an intestinal dysbiosis caused by a Dialister spp. to Phascolarctobacterium spp. (D/P) ratio superior to 1 and/or suffering from a disease or disorder associated with an intestinal dysbiosis selected from the group consisting of IBD, particularly ulcerative colitis or Crohn's disease.

The invention then also concerns a kit of parts comprising i) an agent able to decrease Dialister spp. population and ii) an agent able to increase Phascolarctobacterium spp. population as a combined preparation for simultaneous, separate or sequential use, in particular for use in the treatment of a subject having a Dialister spp. to Phascolarctobacterium spp. ratio associated with a subject at risk of developing inflammatory bowel disease (IBD) or suffering from IBD, in particular having a D/P ratio higher than 1.

Preferably, the agent able to decrease Dialister spp. population is an antibiotic targeting anaerobic gut bacteria, preferably Firmicutes, more preferably Tenericutes for instance Vancomycin or laxatives, or salts to modify transit. Other suitable agents include phages specific to genus or strain and/or substrate scavenging agents and the agent able to increase Phascolarctobacterium spp. population is a composition comprising Phascolarctobacterium spp. Other suitable agents include targeted prebiotics and active modulating agents of intestinal pH, RedOx and/or substrate avail. Methods to identify suitable phages are known to the skilled person.

The present invention also relates to a kit comprising detecting means for determining the ratio of Dialister spp. To Phascolarctobacterium spp. More particularly, the detecting means are suitable for determining the amount of Dialister spp. and the amount of Phascolarctobacterium spp. Optionally, the detecting means are suitable for determining the total number, percentage, abundance or concentration of Dialister spp. and of Phascolarctobacterium spp.

The present invention also relates to a kit comprising detecting means designed to specifically detect i) Dialister spp. and Phascolarctobacterium spp., and ii) optionally Prevotella spp., the means being preferably primer sets or probe(s) specific for detecting Dialister spp. and primer sets or probe(s) specific for detecting Phascolarctobacterium spp., and optionally primer sets or probe(s) specific for detecting Prevotella spp.

Such kit preferably comprises primer sets or probes designed to specifically hybridize the hypervariable regions of the 16S rRNA gene of Dialister spp. and Phascolarctobacterium spp., respectively, and optionally primer sets or probes designed to specifically hybridize the hypervariable regions of the 16S rRNA gene of Prevotella spp., preferably to the V3/V4 region of the hypervariable regions of the 16S rRNA genes.

Particularly, the detecting means can be a primer set and/or probe specific for Dialister spp. and a primer set and/or probe specific for Phascolarctobacterium spp. The kit comprises primer set or probe which specifically hybridizes to the 16S rRNA gene of Dialister spp. and primer set or probe which specifically hybridizes to the 16S rRNA gene of Phascolarctobacterium spp., especially the hypervariable regions of the 16S rRNA gene of Dialister spp. And Phascolarctobacterium spp. More specifically, the kit comprises primer set or probe which specifically hybridizes to a 16S rRNA sequence having at least 85%, at least 90%, at least 95%, at least 99% or at least 100% identity with any one of SEQ ID Nos: 1, 2, 3, 4, 5, 6 and 7, and primer set or probe which specifically hybridizes to a 16S rRNA sequence having at least 85%, at least 90%, at least 95%, at least 99% or at least 100% identity with any one of SEQ ID Nos: 8, 9 and 10. Optionally, the primer set or probe hybridize with a medium/high stringency, preferably a high stringency. Even more particularly, the kit comprises a primer set suitable for amplifying the V3/V4 region of the hypervariable regions of the 16S rRNA gene. Optionally, the kit may comprise sequencing means such as Illumina MiSeq.

In a very specific aspect, the kit comprises a primer set including 5′-GAGTATCGGAGAGGAAAGTGGA-3′ (SEQ ID No: 11) and 5′-GCTTCTCTTTGTTGACACCCATT-3′ (SEQ ID No: 12) for Dialister spp. and 5′-GGAGTGCTAATACCGGATGTGA-3′ (SEQ ID No: 13) and 5′-CCGTGGCTTCCTCGTTTACT-3′ (SEQ ID No: 14) for Phascolarctobacterium spp. Additionally, the kit may comprise a primer set including 5′-CACRGTAAACGATGGATGCC-3′ (SEQ ID No: 17) and 3′-CCAGACGTTGGGCTGG-5′ (SEQ ID No: 18) for Prevotella spp.

The present invention finally relates to the use of the kit as described above for detecting a ratio of Dialister spp. to Phascolarctobacterium spp. or a ratio of Phascolarctobacterium spp. to Dialister spp. and optionally the presence of Prevotella spp., in particular in a biological sample from a subject, preferably a stool sample from a subject.

The present invention finally relates to the use of the kit as described above for diagnosis purpose of an intestinal dysbiosis, or of a disease or a disorder associated with an intestinal dysbiosis selected from the group consisting of IBD, particularly ulcerative colitis or Crohn's disease, colorectal cancer, atherosclerotic cardiovascular disease and low body mass index, preferably IBD, for stratifying patient population suffering from such disease or disorder in a clinical trial, for selecting a subject suffering from such diseases or disorder that will benefit from a treatment, or for monitoring treatment response in subject suffering from such diseases or disorder.

The present invention finally relates to the use of the kit as described above for detecting a ratio of Dialister spp. To Phascolarctobacterium spp. or the ratio of Phascolarctobacterium spp. To Dialister spp. and optionally the presence of Prevotella spp. in a stool sample from a subject, in particular for diagnosis purpose of a disease or disorder associated with dysbiosis, preferably selected from the group consisting of inflammatory bowel disease (IBD), colorectal cancer, atherosclerotic cardiovascular disease and low body mass index (underweight), preferably inflammatory bowel disease (IBD), for stratifying patient population in a clinical trial, or for monitoring treatment response.

### EXAMPLES

### Example 1: Human Gut Microbiota Differ in their Ability and Rate to Metabolize Succinate In Vitro

In order to obtain an overview of the potential differences in succinate consumption by complex intestinal microbiota, the inventors performed in vitro enrichments of feces from 10 different human donors. To this end, they diluted the fecal samples in a base medium supplemented with 30 mM of succinate, and measured the residual amount of succinate and produced short-chain fatty acids (SCFA) after 2 and 7 days of strict anaerobic cultivation. Despite identical cultivation conditions, the 10 fecal microbiota differed in the rate at which they consumed the supplied succinate (FIG. 1A). Some microbiomes were able to consume all or the majority of the succinate within the first 2 days in at least one replicate, while others did not manage to convert all the succinate even after seven days.

### Example 2: The Consumption of Succinate by Fecal Microbiota is Associated with the Bacterial Genera Phascolarctobacterium, Phascolarctobacterium_A, and Dialister

To determine whether the differences in succinate consumption rate is due to differences in microbiota composition, the inventors performed 16S amplicon sequencing of the enrichment cultures of both the SU and SU-cultures. They computed the ‘succinate-specific enrichment’ as the difference in relative abundance of a genus after growth in SU and SU-. Doing so, they identified the top three genera as specifically enriched in succinate conditions (FIG. 1B). Interestingly, some of these genera appeared to be specifically associated with the rates of succinate consumption. Phascolarctobacterium and Phascolarctobacterium_A were predominantly enriched in the fast microbiota, and Dialister in slow microbiota.

### Example 3: Intestinal Succinate-Consuming Bacteria Differ in their Succinate Conversion Rate

The inventors performed single-culture growth experiments of bacterial isolates belonging to the genera Phascolarctobacterium and Dialister. They inoculated representative isolates in parallel pure cultures of quasi-equal number of viable cells in a base medium supplemented with 80 mM of succinate.

They measured the amount succinate consumed and SCFA produced at different time intervals up to 10 days (FIG. 6A). All cultures showed an exponential increase in consumed succinate over time but differed in the rate of succinate conversion. They estimated the culture-level consumption rate by linear regression of the log-transformed succinate concentration. Phascolarctobacterium_A succinatutens had the highest consumption rate with 0.105 mM/h, followed by Phascolarctobacterium faecium with 0.100 mM/h, Dialister hominis with 0.063 mM/h, and Dialister invisus with 0.046 mM/h (FIG. 6B).

### Example 4: Human Fecal Microbiomes can be Stratified by their ‘Succinotype’

The enrichment data of fecal microbiomes suggest that only a single species dominates the succinate-consumer niche at a time the enriched fecal samples. To test whether this is also more generally true in human fecal microbiomes, the inventors analyzed four publicly available cohorts of 16S amplicon intestinal microbiome data with a total of 10,169 samples. They determined the relative abundance of 16S amplicons assigned to either the genus Phascolarctobacterium (containing Phascolarctobacterium and Phascolarctobacterium_A) or Dialister.

The vast majority of samples had detectable abundances of either Dialister or Phascolarctobacterium (9,670/10,169), supporting the notion that these two genera represent the common succinate utilizers in human microbiomes. Furthermore, Dialister and Phascolarctobacterium were highly mutually exclusive across all cohorts (FIG. 2). Of those samples with sufficient reads to robustly compute a relative abundance, over 85% of the samples (7,519/8,830) had a difference in relative abundance between the two genera of over one order of magnitude. Based on this strong signal, the inventors defined the ‘succinotype’ of a sample as either ‘D’ or ‘P’ depending on whether Dialister or Phascolarctobacterium was the dominant member.

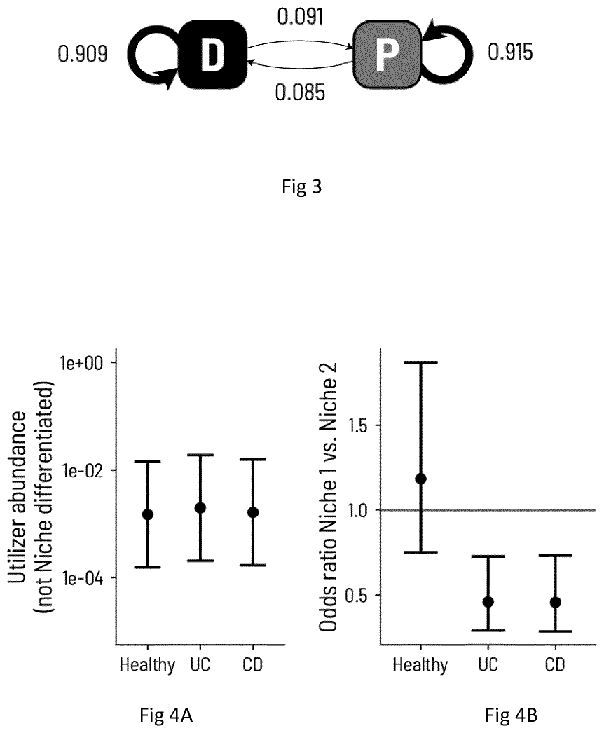

Individuals maintained their succinotype over time. The inventors computed the probability that an individual transitions from a D-to-P succinotype (or vice versa) from the UCC cohort. Individuals with a D and P succinotype had the same one in the subsequent fecal sample 90.1% and 91.5% of the time, respectively (FIG. 3). Thus, succinotypes are robustly associated with individuals, and hence that the measured differences in succinate consumption rate might imply that individuals with a P succinotype remove succinate more rapidly than those with a D succinotype.

### Example 5: Patients with IBD are More Likely to have a Dialister Succinotype than Healthy Individuals

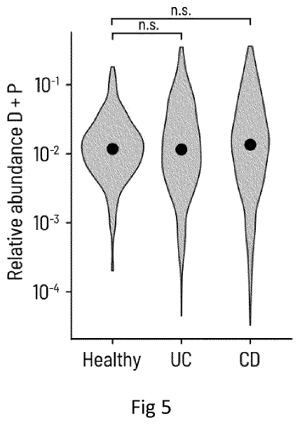

The inventors compared the distribution of succinotypes within the cohorts that compared healthy individuals with UC and Crohn's Disease (CD) patients. Patients suffering from UC or Crohn's disease were significantly more likely to have a D succinotype than a P succinotype The odds ratio of P-to-D was 0.453 (CI=[0.289,0.711], z=−3.44, p=0.000572) and 0.451 (CI=[0.284,0.717], z=−3.37, p=0.000751) for UC and CD, respectively (FIG. 4B). Notably, the total relative abundance of succinate consumers irrespective of succinotype did not significantly differ between UC or CD patients and healthy individuals (FIG. 5). Hence, the association between succinotype and UC/CD could not be explained simply by differences in succinate consumer abundance. This suggests that the identity of the dominant succinate consumer is the main contributor to the observed differentiation between patients and healthy individuals.

### Example 6: Dialister/Phascolarctobacterium Ratio is Indicative for a Range of Diseases and Physical Conditions, Including IBD, CRC, ACVD (Atherosclerotic Cardiovascular Disease) and Low BMI

The inventors compared the distribution of succinotypes within various cohorts across different diseases from the publicly available dataset curatedMetagenomicData (see for example Pascolli et al., Nature Methods volume 14, pages 1023-1024 (2017)). Succinotypes were defined for individuals based on the relative abundance of taxa that belonged to either the genus Dialister or Phascolarctobacterium. Individuals for whom neither genus were detected were excluded from the analysis. The inventors then analyzed three diseases with respect to the number of patients across cohorts: inflammatory bowel disease (IBD), colorectal cancer (CRC) and atherosclerotic cardiovascular disease (ACVD). For each disease, a logistic regression was performed to estimate the change in log-odds of disease versus healthy for individuals with a Phascolarctobacterium succinotype relative to those with a Dialister succinotype. In IBD, having a Phascolarctobacterium succinotype decreased the log-odds of having IBD by −0.176 (CI=[−0.322, −0.029], z=−2.35, p=0.0187) compared to the health individuals in the respective cohorts. These results using data from shotgun metagenomic sequencing is in agreement with the results obtained in other cohorts using data from 16S amplicon sequencing. In CRC, having a Phascolarctobacterium succinotype increased the log-odds of having disease by 0.231 (CI=[0.00295, 0.459], z=1.99, p=0.0471). In ACVD, having a Phascolarctobacterium succinotype increased the log-odds of having disease by 0.670 (CI=[0.331, 1.01], z=3.86, p=0.000113).

The inventors also compared the distribution of succinotypes within the American Gut Project cohort as a function of their body mass index (BMI). The odds ratio of P-to-D in Individuals with a normal BMI (18.5<BMI<25) was 1.38 (CI=[1.30,1.46], z=10.7, p<1e-10). The odds ratio of P-to-D for overweight (25<BMI<30) and obese individuals (BMI>30) was 1.33 (CI=[1.21,1.46], z=6.05, p=1.49e-9) and 1.44 (CI=[1.25,1.66], z=4.98, p=6.39e-7), respectively. These odds ratios were not significantly different from those for individuals with a normal BMI. Underweight individuals (BMI<18.5) had a P-to-D odds ratio of 0.97 (CI=[0.83, 1.13], z=−0.39, p=0.70). The odds ratio for underweight individuals was significantly lower than for normal individuals (glm, z=−4.18, p=0.0000288).

### Example 7: The Informative Power of the D/P Biomarker can be Improved by Testing Whether a Subgroup Of Succinotypes has a Microbiome Comprising Strains of the Prevotella Genus

The corkIBD cohort (see for example Clooney et al., Gut 2021; 70:499-510) was used to characterize how Ulcerative colitis (UC) prevalence compares among subgroups of the cohorts. The proportions of the disease state in the individual samples (UC, n=250; healthy, n=160) was compared based on the identity of the dominant succinate utilizer: Phascolarctobacterium or Dialister. In addition to the dominant succinate utilizer, the presence or absence of Prevotella enabled further stratification of the individuals. Without any stratifications, the incidence of UC in this recruited cohort was 61%. Stratification based on succinotype alone shows that the Dialister-type subpopulation has increased UC incidence at 71.2% compared to the Phascolarctobacterium-type subpopulation has decreased UC incidence at 45.3%. Furthermore, Dialister-type individuals that did not have detectable Prevotella had the highest incidence at 77.4%, while Phascolarctobacterium-type individuals with detectable Prevotella had the lowest incidence of UC at 37.7%.

### Methods

Preparation of anaerobic culture media. Media for in vitro enrichments of complex cultures were adapted from M2GSC medium, and media to characterize single strains were either adapted from M2GSC or YCFA medium. All media were prepared following the same protocol: All medium ingredients except sodium bicarbonate and L-cysteine HCl were dissolved in an Erlenmeyer flask and the pH was adjusted to pH 7 using sodium hydroxide (5 mM). The media were boiled for 15 min for major removal of oxygen, under constant moderate stirring and using a Liebig condenser to prevent vaporization of ingredients. After boiling, the media were constantly flushed with CO2. Sodium bicarbonate and L-cysteine hydrochloride monohydrate were added when the media cooled down to 55° C. for further reduction of residual oxygen for 10 min. Aliquots of 8 mL of medium were filled into Hungate tubes under constant flushing with CO2, and Hungate tubes were sealed with butyl rubber stoppers and screw caps (Millan SA, Geneva, Switzerland). Media were sterilized by autoclaving and stored at room temperature.

Fecal Inocula. Fresh fecal samples were donated from 10 healthy individuals with no history of antibiotic use, intestinal infections, or severe diarrhea during the three months prior to making the donation. The donors did not take immunosuppressive drugs, blood thinners, or medication affecting the bowel passage or digestion. Fecal samples were anaerobically transported in an airtight container together with an Oxoid™ AnaeroGen™ 2.5 L sachet (Thermo Fisher Diagnostics AG, Pratteln, Switzerland) and processed within three hours after defecation. Total viable cells after transport were determined by MPN enumeration in liquid culture and using strict anaerobic Hungate techniques, with an accepted range of 10¹⁰-10¹² viable cells per gram of feces as expected for healthy stool. Stool consistency was evaluated optically according to the Bristol Stool Scale and samples within the defined range of a healthy stool, notably with a score between 3-5, were accepted.

For processing, the fecal samples were transferred into an anaerobic chamber (10% CO2, 5% H2, and 85% N2) (Coy Laboratories, Ann Arbor, MI, USA). One gram of fecal sample measured with a sterile plastic spoon (VWR International, Dietikon, Switzerland) was suspended in 9 mL of anaerobic dilution solution in a sterile 50 mL Falcon tube and using a 25 ml sterile serum pipet. The dilution step was repeated and 1 mL of the 10⁻² dilution was transferred into a sterile Hungate tube containing 9 mL of ADS. Serial dilutions down to 10⁻¹¹ were continued outside of the anaerobic chamber under sterile, anaerobic conditions using Hungate technique.

Batch enrichments in succinate-rich conditions. Anaerobic in vitro enrichments were performed in Hungate tubes sealed with butyl rubber stoppers and screw caps (Millan SA, Geneva, Switzerland). For each enrichment, 0.3 mL of the fecal sample dilution 108 was inoculated into 8 mL of cultivation medium under sterile, anaerobic conditions using Hungate technique. Enrichments were performed for each of the 10 studied microbiota in three replicate cultures in succinate-rich conditions, i.e. a basal medium supplemented with 30 mM of disodium succinate), and in a control condition, i.e. non-supplemented basal medium. Media were buffered at an initial pH of 6.5. All cultures were incubated at 37° C. and the optical density, the metabolite profile and the composition of the microbial community were determined after two and seven days of cultivation.

Succinate-specific enrichment score. The genus-level composition of each of the SU and SU-enrichment cultures based on 16S amplicon sequencing was determined. The inventors computed the genus-level relative abundance by grouping all ASVs that were taxonomically classified as the same genus.

The inventors determined the background growth on the base medium as the mean relative abundance of those cultures with non-zero read counts. The inventors excluded zero-count cultures as these genera were likely lost during the inoculum dilution. The inventors then computed the succinate-specific enrichment as the difference in relative abundance of the genus in the SU cultures minus the background relative abundance on SU-.

Determination of the succinate consumption rate of confirmed succinate-consuming strains. All single strains were pre-cultured in YCFA medium supplemented with 80 mM of succinate. For precultures used as inocula for the succinate consumption tests, viable cell counts were determined by flow cytometry using live/dead staining (see protocol below). Precultures with higher viable cell counts were first diluted in anaerobic dilution solution to inoculate a target cell concentration of 107, and specifically of 4.2E+06 to 4.8E+07 viable cells in 0.1 mL. All cultures to determine the succinate consumption rate were performed in 8 mL of YCFA medium supplemented with 80 mM of succinate. Different sampling intervals were set for different succinate-consuming taxa based on preliminary tests and with the target of 20 sampling points throughout the growth period on succinate: Intervals of 1 h for a maximum duration of 25 h for Phascolarctobacterium; and intervals of 4 h for a maximum of 100 h for Dialister. Individual cultures were performed for all sampling points to avoid impacts on the succinate consumption rate by repeated sampling procedure. Individual cultures were performed from two different inoculum cultures (n=2) for all remaining strains. The metabolite profile and the optical density as a readout for biomass production of all cultures were measured at each sampling point.

Metabolite analysis. SCFA concentrations (formate, acetate, propionate, butyrate, and valerate), branched-chain fatty acids (BCFAs) (isobutyrate and isovalerate) and intermediate metabolites (lactate, succinate, and ethanol) were measured by HPLC analysis. Hit Analyses were performed with a Hitachi Chromaster 5450 RI-Detector (VWR International GmbH, Schlieren, Switzerland) using a Rezex ROA-Organic Acid (4%) precolumn connected to a Rezex ROA-Organic Acid (8%) column, equipped with a Security Guard Carbo-H cartridge (4×3.0 mm). Samples were prepared from 1 ml of bacterial culture centrifuged at 14′000 g for 10 min at 4° C. The supernatant was filtered into 2 mL short thread vials with crimp caps (VWR International GmbH, Schlieren, Switzerland) using non-sterile 0.2 μm regenerated cellulose membrane filters (Phenomenex Inc., Aschaffenburg, Germany). A volume of 40 ul of sample was injected into the HPLC with a flow rate of 0.6 mL/min at a constant column temperature of 80° C. and H2SO4 (10 mM) and Na-azide (0.05 g/L) as eluent. Metabolite concentrations were determined using external standards (all purchased from Sigma-Aldrich, Buchs, Switzerland). Peaks were integrated using the EZChromElite software (Version V3.3.2.SP2, Hitachi High Tech Science Corporation).

DNA extractions and compositional profiling by 16S metagenomic sequencing. The total genomic DNA was extracted from 200 mg of fecal samples and from pellets of 1 mL culture samples (centrifuged at 14′000 g for 10 min at 4° C.), according to a fecal sample-based adaptation to the Maxwell® RSC PureFood GMO and Authentication Kit (Catalog No. AS1600; Promega Corporation, Dübendorf, Switzerland). Quality of all DNA extracts was confirmed on a Tris-Acetate-EDTA (TAE)-1.5% agarose gel. The total DNA concentration after extraction was quantified using the Qubit® dsDNA HS Assay kit (Thermo Fisher Scientific, Pratteln, Switzerland). The inventors performed amplicon sequencing of the 16S rRNA V3-V4 region on MiSeq platform (Illumina, CA, USA) using the primer combination 341F (5″-CCTACGGGNBGCASCAG-3″, SEQ ID No: 15)) and 806bR (5″-GGACTACNVGGGTWTCTAAT-3″, SEQ ID No: 16). Library preparation and sequencing was performed by StarSEQ GmbH (Mainz, Germany) with 25% PhiX to balance the composition of bases. Amplicon Sequence Variants (ASVs) were inferred using Dada2 v1.18.0 with read length filtering set to c (250, 210), maxEE set to c (4,5), and inference done In ‘pseudo pool’ mode. Read pairs were merged with minOverlap of 20, and Bimeras were removed using the ‘consensus’ method. The prepared GTDB r95 taxonomic database for Dada2 (Alishum and Others, 2019) was used for taxonomic annotations via the ‘assignTaxonomy’ function in Dada2.

Determination of viable cell counts by flow cytometry. Viable cell counts of bacterial cultures used as inocula for succinate consumption tests were determined using live/dead staining followed by flow cytometry. A double staining assay with the two nucleic acid dyes SYBR Green (SG) and propidium iodide (PI) was used to differentiate between cells with intact (viable) and damaged (dead) cytoplasmic membranes. The SG+PI staining solution was prepared by mixing 970 μl of DMSO with 10 μL of SYBR Green 10,000× stock solution (Catalog No. S7563, Thermo Fisher Scientific, Pratteln, Switzerland) and 20 μl of a 20 mM PI (Catalog No. PI304MP, Thermo Fisher Scientific, Pratteln, Switzerland) stock solution in DMSO. Bacterial cultures were diluted 10′000-fold in filtered (0.20 μM) anaerobic dilution solution (ADS) without added resazurin, cysteine and bicarbonate to obtain an appropriate bacterial culture for flow cytometry. For stainings, 1000-fold diluted samples of 30 μL were added to 267 μl of filtered (0.20 μM) ADS without added resazurin, cysteine and bicarbonate to obtain a final 10,000-fold sample dilution in a reaction volume of 300 μL. 3 μL of the SGPI stock solution were added followed by immediate mixing by vortexing. Samples were stained for 20 min at room temperature protected from light. Flow cytometry was performed using a Guava® easyCyte HT device (Merck, Schaffhausen, Switzerland) and the InCyte software version 3.3. For each series of measurements, a medium control was included to exclude false positive staining.

1.-37. (canceled)

38. A method for assessing whether a subject is suffering or is at risk of developing a disease or disorder associated with an intestinal dysbiosis, comprising:
a) providing a biological sample from the subject;
b) determining a ratio of Dialister spp. to Phascolarctobacterium spp. (D/P) or a ratio of Phascolarctobacterium spp. to Dialister spp. (P/D) in the biological sample; and
c) classifying the subject as suffering or being at risk of developing the disease or disorder associated with an intestinal dysbiosis based on the determined ratio of Dialister spp. to Phascolarctobacterium spp. or the determined ratio of Phascolarctobacterium spp. to Dialister spp.

39. The method according to claim 38, wherein the disease or disorder associated with an intestinal dysbiosis is inflammatory bowel disease (IBD), and wherein the subject is classified as suffering or being at risk of developing inflammatory bowel disease (IBD) if the ratio D/P is superior to 1 or the ratio P/D is inferior to 1.

40. The method according to claim 38, wherein the method further comprises the determination of the presence or absence of Prevotella spp. in the biological sample.

41. The method according to claim 40, wherein the disease or disorder associated with an intestinal dysbiosis is inflammatory bowel disease (IBD), and wherein the subject is classified as suffering or being at risk of developing inflammatory bowel disease (IBD) if the ratio D/P is greater than 1 or the ratio P/D is less than 1, and Prevotella is absent.

42. The method according to claim 38, wherein the ratio of Phascolarctobacterium spp. to Dialister spp. is the result of dividing total number, percentage, abundance or concentration of Phascolarctobacterium spp. by total number, percentage, abundance or concentration of Dialister spp., respectively.

43. The method according to claim 42, wherein the abundance is measured using optical density, qPCR, microarray technique, amplicon sequencing, single cell sequencing or single cell Matrix Assisted Laser Desorption Ionization-Time of Flight (MALDI-TOF) following flow cytometry or other microfluidic methods, chamber counting, total bacterial DNA quantification or metagenomic sequencing and grouping of genes, proteomic profiling or total RNA sequencing of complex samples.

44. The method according to claim 42, wherein the abundance is measured by amplifying the V3/V4 region of the hypervariable regions of the 16S rRNA gene and by sequencing amplicons.

45. The method according to claim 38, wherein the biological sample is a stool sample.

46. A method for monitoring treatment response in a subject suffering of a disease or disorder associated with an intestinal dysbiosis, comprising:
a) providing a first biological sample from the subject at a first time;
b) determining a first ratio of Dialister spp. to Phascolarctobacterium spp. in the first biological sample;
c) providing a second biological sample from the subject at a second time, the second time being after the first time;
d) determining a second ratio of Dialister spp. to Phascolarctobacterium spp. in the second biological sample; and
e) comparing the first ratio and the second ratio.

47. The method according to claim 46, wherein the disease or disorder associated with an intestinal dysbiosis is inflammatory bowel disease (IBD), and wherein a decrease of the ratio is indicative of an effective treatment of IBD in the subject.

48. A method for monitoring treatment response in a subject suffering of a disease or disorder associated with an intestinal dysbiosis, comprising:
a) providing a first biological sample from the subject at a first time;
b) determining a first ratio of Phascolarctobacterium spp. to Dialister spp. in the first biological sample;
c) providing a second biological sample from the subject at a second time, the second time being after the first time;
d) determining a second ratio of Phascolarctobacterium spp. to Dialister spp. in the second biological sample; and
e) comparing the first ratio and the second ratio.

49. A method for selecting a subject suffering from a disease or disorder associated with an intestinal dysbiosis as susceptible to benefit from a treatment comprising the administration of Phascolarctobacterium spp., wherein the method comprises:
a) providing a biological sample from the subject;
b) determining a ratio of Dialister spp. to Phascolarctobacterium spp. (D/P) or a ratio of Phascolarctobacterium spp. to Dialister spp. (P/D) in the biological sample; and
c) selecting the subject based on the determined ratio of Dialister spp. to Phascolarctobacterium spp. (D/P) or Phascolarctobacterium spp. to Dialister spp. (P/D) as susceptible to benefit from the treatment.

50. A kit comprising detecting means designed to specifically detect i) Dialister spp. and Phascolarctobacterium spp., ii) optionally Prevotella spp., the means being primer sets or probe(s) specific for detecting Dialister spp. and primer sets or probe(s) specific for detecting Phascolarctobacterium spp., and optionally primer sets or probe(s) specific for detecting Prevotella spp.

51. The kit according to claim 50, wherein the kit comprises primer sets or probes designed to specifically hybridize the hypervariable regions of the 16S rRNA gene of Dialister spp. and Phascolarctobacterium spp., respectively, and optionally primer sets or probes designed to specifically hybridize the hypervariable regions of the 16S rRNA gene of Prevotella spp.


SEQUENCE LISTING (18 sequences)

SEQ ID NO: 1 - DNA, 1565 bp, synthetic construct
  misc_feature 1..1565: note = Dialister
  source 1..1565: mol_type = other DNA
aggaggtgatccagccgcaccttccgatacggctaccttgttacgacttcactccaatcatcggctccaccttcgacggctggctccttgcggttacctcaccggcttcgggtgtttccgactctcatagtgtgacgggcggtgtgtacaaggcccgggaacgtattcaccgcggtatgctgacccgcgattactagcgattcctacttcatgcaggcgggttgcagcctgcaatctgaactgggggtgtgtttttgaggttcgctccacctcgcggtttcgcttctctttgttgacacccattgtagtacgtgtgtagcccaggtcataaggggcatgatgacttgacgtcatccccaccttcctccgcgttgtccgcggcggtctctcatgagtccccaccataacgtgctggtaacataagataggggttgcgctcgttgcgggacttaacccaacatctcacgacacgagctgacgacagccgtgcaccacctgttttctcgtctcccgaaggagaacttccaatttcttggaatagcgatcaatgtcaagacctggtaaggttcttcgcgttgcgtcgaattaaaccacatactccaccgcttgtgcgggcccccgtcaattcctttgagttttaatcttgcgatcgtacttcccaggcggaatacttattgtgttagctccggcacagaaggggtcgatacctcctacacctagtattcatcgtttacggcgtggactaccagggtatctaatcctgtttgctccccacgctttcgcgcctcagcgtcagttgtcgtccagaaagtcgccttcgccaccggtgttcttcctaatctctacgcatttcaccgctacactaggaattccactttcctctccgatactccagcttcccagtttccatcccatcacggggttaagccccgcacttttaagatggacttaggaagccgcctgcgcgcgctttacgcccaataattccggacaacgcttgccacctacgtattaccgcggctgctggcacgtagttagccgtggcttgctatccgggtaccgtcacttctataagttattggcttacagaccgttcgtccccggtcacagagctttacaacccgaaggccgtcatcactcacgcggcgttgctccgtcaggctttcgcccattgcggaagattccccactgctgcctcccgtaggagtctggaccgtgtctcagttccagtgtggccgttcatcctctcagaccggctactgatcgtaggtttggtgggccgttacctcaccaactgcctaatcagacgcaaacccctcttcaggcgatagcttacaggtagaggccaccctttccttggaaaatcatgcggtcttccaagcgtattcggtattagcagtcgtttccgtctgttgtccccatcctgaaggcaggttgtttacgtgttactcacccgtttgccactcgatttcaaaagaagcaagcttctcttgtcctctcgttcgacttgcatgtgttaagcacgccgccagcgttcgtcctgagccaggatcaaactctccatg

SEQ ID NO: 2 - DNA, 1541 bp, synthetic construct
  misc_feature 1..1541: note = Dialister
  source 1..1541: mol_type = other DNA
ggagagtttgatcctggctcaggacgaacgctggcggcgtgcttaacacatgcaagtcgaacgagaagagaaggaagcttgcttctttcaaatctagtggcaaacgggtgagtaacacgtaaacaacctgccttcaggatggggacaacagacggaaacgactgctaataccgaatacgttccttaggtcgcatgatcttgggaagaaagggtggcctctacctgtaagctatcgcctgaagaggggtttgcgtctgattaggtagttggtgaggtaacggcccaccaagccgacgatcagtagccggtctgagaggatgaacggccacactggaactgagacacggtccagactcctacgggaggcagcagtggggaatcttccgcaatgggcgaaagcctgacggagcaacgccgcgtgagtgatgacggccttcgggttgtaaagctctgtgatcggggacgaacggtcggcagacgaataatctgctgaattgacggtacccgaatagcaagccacggctaactacgtgccagcagccgcggtaatacgtaggtggcaagcgttgtccggaattattgggcgtaaagcgcgcgcaggcggcttcttaagtccatcttaaaagtgcggggcttaaccccgtgatgggatggaaactgagaggctggagtatcggagaggaaagtggaattcctagtgtagcggtgaaatgcgtagagattaggaagaacaccggtggcgaaggcgactttctggacgacaactgacgctgaggcgcgaaagcgtggggagcaaacaggattagataccctggtagtccacgccgtaaacgatgaatactaggtgtaggaggtatcgaccccttctgtgccggagctaacacaataagtattccgcctgggaagtacgatcgcaagattaaaactcaaaggaattgacgggggcccgcacaagcggtggagtatgtggtttaattcgacgcaacgcgaagaaccttaccaggtcttgacattgatcgctattccaagaaattggaagttcctcttcggaggacgagaaaacaggtggtgcacggctgtcgtcagctcgtgtcgtgagatgttgggttaagtcccgcaacgagcgcaacccctatcttatgttaccagcacgttatggtggggactcatgagagaccgccgcggacaacgcggaggaaggtggggatgacgtcaagtcatcatgccccttatgacctgggctacacacgtactacaatgggtgtcaacaaagagaagcgaagccgcgaggcagagcaaacctcaaaaacacacccccagttcagattgcaggctgcaactcgcctgcatgaagtaggaatcgctagtaatcgcgggtcagcataccgcggtgaatacgttcccgggccttgtacacaccgcccgtcacactatgagagtcagaaacacccgaagccggtgaggtaaccgcaaggagccagccgtcgaaggcggagctgatgattggagtgaagtcgtaacaaggtagccgtatcggaagg

SEQ ID NO: 3 - DNA, 1565 bp, synthetic construct
  misc_feature 1..1565: note = Dialister
  source 1..1565: mol_type = other DNA
aggaggtgatccagccgcaccttccgatacggctaccttgttacgacttcactccaatcatcagctccgccttcgacggctggctccttgcggttacctcaccggcttcgggtgtttctgactctcatagtgtgacgggcggtgtgtacaaggcccgggaacgtattcaccgcggtatgctgacccgcgattactagcgattcctacttcacgcaggcgggttgcagcctgcgatctgaactgggggtgtgtttttgaggtttgctcttcctcgcgggttcgcttctctttgttgacacccattgtagtacgtgtgtagcccaggtcataaggggcatgatgacttgacgtcgtccccgccttcctccgcgttgtccgcggcggtctcatttgagtccccacctttacgtgctggtaacaaatgataggggttgcgctcgttgcgggacttaacccaacatctcacgacacgagctgacgacagccgtgcaccacctgttttctcgtcttccgaagaagaactccgcatttctgcagatcgcgatcaatgtcaagacctggtaaggttcttcgcgttgcgtcgaattaaaccacatactccaccgcttgtgcgggcccccgtcaattcctttgagttttaatcttgcgatcgtacttcccaggcgggatacttattgcgttaactccggcacaggaggggtcgatacctcctacacctagtatccatcgtttacggcgtggactaccagggtatctaatcctgtttgctccccacgctttcgcgcctcagcgtcagttttcgtccagaaagtcgccttcgccaccggtgttcttcctaatctctacgcatttcaccgctacactaggaattccactttcctctccgatactccagcttcccagtttccttcccatcacggggttaagccccgcacttttaagagggacttgggaagccgcctgcgcgcgctttacgcccaataattccggacaacgcttgccacctacgtattaccgcggctgctggcacgtagttagccgtggcttgcttttccggtaccgtcaatgcagtttgttcttcgcactctgccttttcgtcccggatcacagagttttacaacccgaaggccgtcatcactcacgcggcgttgctccgtcaggctttcgcccattgcggaagattccccactgctgcctcccgtaggagtctggaccgtgtctcagttccagtgtggccgttcatcctctcagaccggctactgatcgtcgccttggtgggccgttacccctccaaccagctaatcagacgcaaacccctcttcaggcgatagcttacaggtagaggccaccttttcttccatgggccatgcggctcttggaacttattcggtattagcagtcgtttccgtctgttgtccccatcctgaaggcaggttgtttacgtgttactcacccgtttgccactcaattccggaaagagcaagctctttccctcttttcgttcgacttgcatgtgttaagcacgccgccagcgttcgtcctgagccaggatcaaactctccatg

SEQ ID NO: 4 - DNA, 1563 bp, synthetic construct
  misc_feature 1..1563: note = Dialister
  source 1..1563: mol_type = other DNA
ggagagtttgatcctggctcaggacgaacgctggcggcgtgcttaacacatgcaagtcgaacgagaggacatgaaaagcttgctttttatgaaatctagtggcaaacgggtgagtaacacgtaaacaacctgccttcaagatggggacaacagacggaaacgactgctaataccgaatacgatccgaaagtcgcatgacatttggatgaaagggtggcctatcgaagaagctatcgcttgaagaggggtttgcgtccgattaggtagttggtgaggtaacggcccaccaagccgacgatcggtagccggtctgagaggatgaacggccacactggaactgagacacggtccagactcctacgggaggcagcagtggggaatcttccgcaatggacgaaagtctgacggagcaacgccgcgtgagtgaagacggccttcgggttgtaaagctctgtgattcgggacgaaaggccatatgtgaataatatatggaaatgacggtaccgaaaaagcaagccacggctaactacgtgccagcagccgcggtaatacgtaggtggcaagcgttgtccggaattattgggcgtaaagcgcgcgcaggcggtcacttaagtccatcttagaagtgcggggcttaaccccgtgatgggatggaaactgggagactggagtatcggagaggaaagtggaattcctagtgtagcggtgaaatgcgtagatattaggaagaacaccggtggcgaaggcgactttctggacgaaaactgacgctgaggcgcgaaagcgtggggagcaaacaggattagataccctggtagtccacgccgtaaacgatggatactaggtgtaggaggtatcgaccccttctgtgccggagttaacgcaataagtatcccgcctgggaagtacgatcgcaagattaaaactcaaaggaattgacgggggcccgcacaagcggtggagtatgtggtttaattcgacgcaacgcgaagaaccttaccaagtcttgacattgatcgccattccaagagattggaagttctccttcgggagacgagaaaacaggtggtgcacggctgtcgtcagctcgtgtcgtgagatgttgggttaagtcccgcaacgagcgcaacccctatcttttgttgccagcacgtagaggtgggaactcagaagagaccgccgcagacaatgcggaggaaggtggggatgacgtcaagtcatcatgccccttatgacttgggctacacacgtactacaatgggctttaacaaagagcagcgaaaccgcgaggtggagcgaaactcaaaaacaagcccccagttcagatcgcaggctgcaactcgcctgcgtgaagcaggaatcgctagtaatcgcgggtcagcataccgcggtgaatacgttcccgggccttgtacacaccgcccgtcacactatgagagtcggaaacacccgaagccggtgaggtaaccgcaaggagccagccgtcgaaggtggggctgatgattggagtgaagtcgtaacaaggtagccgtatcggaaggtgcggctggatcacctcctttc

SEQ ID NO: 5 - DNA, 1565 bp, synthetic construct
  misc_feature 1..1565: note = Dialister
  source 1..1565: mol_type = other DNA
aggaggtgatccagccgcaccttccgatacggctaccttgttacgacttcactccaatcatcagctccaccttcgacggctggctccttgcggttacctcaccggcttcgggtgtttccgactctcatagtgtgacgggcggtgtgtacaaggcccgggaacgtattcaccgcggtatgctgacccgcgattactagcgattcctacttcatgcaggcgggttgcagcctgcaatctgaactgggggtgtgtttttgaggttcgctccacctcgcggtttcgcttctctttgttgacacccattgtagtacgtgtgtagcccaggtcataaggggcatgatgacttgacgtcatccccaccttcctccgcgttgtccgcggcggtctctcatgagtccccaccataacgtgctggtaacataagataggggttgcgctcgttgcgggacttaacccaacatctcacgacacgagctgacgacagccgtgcaccacctgttttctcgtctcccgaaggagaacttcacatttctgtgaatggcgatcaatgtcaagacctggtaaggttcttcgcgttgcgtcgaattaaaccacatactccaccgcttgtgcgggcccccgtcaattcctttgagttttaatcttgcgatcgtacttcccaggcggaatacttattgtgttagctccggcacagaaggggtcgatacctcctacacctagtattcatcgtttacggcgtggactaccagggtatctaatcctgtttgctccccacgctttcgcgcctcagcgtcagttgtcgtccagaaagtcgccttcgccaccggtgttcttcctaatctctacgcatttcaccgctacactaggaattccactttcctctccgatactccagcttcccagtttccatcccatcacggggttaagccccgcacttttaagatggacttagaaagccgcctgcgcgcgctttacgcccaataattccggacaacgcttgccacctacgtattaccgcggctgctggcacgtagttagccgtggcttgctattcgggtaccgtcacttccataagatattagcttacagaccgttcgtccccgatcacagagctttacaacccgaaggccgtcatcactcacgcggcgttgctccgtcaggctttcgcccattgcggaagattccccactgctgcctcccgtaggagtctggaccgtgtctcagttccagtgtggccgttcatcctctcagaccggctactgatcgtaggtttggtgggccgttacctcaccaactgcctaatcagacgcaaacccctcttcaggcgatagcttacaggtagaggccaccctttccttggaagatcatgcggtctcccaagcgtattcggtattagcagtcgtttccgtctgttgtccccatcctgaaggcaggttgtttacgtgttactcacccgtttgccactcgattccaaaagaagcaagcttctcttgtcctctcgttcgacttgcatgtgttaagcacgccgccagcgttcgtcctgagccaggatcaaactctccatg

SEQ ID NO: 6 - DNA, 1565 bp, synthetic construct
  misc_feature 1..1565: note = Dialister
  source 1..1565: mol_type = other DNA
aggaggtgatccagccgcaccttccgatacggctaccttgttacgacttcactccaatcatcagctccaccttcgacggctggctccttgcggttacctcaccggcttcgggtgtttccgactctcatagtgtgacgggcggtgtgtacaaggcccgggaacgtattcaccgcggtatgctgacccgcgattactagcgattcctgcttcacgcaggcgagttgcagcctgcgatctgaactgggggtgtgtttttgaggtttgctccggatcgctccttcgcttctctttgttgacacccattgtagtacgtgtgtagcccaggtcataaggggcatgatgacttgacgtcgtccccgccttcctccgcattgtctgcggcggtctcatttgagttcccacctttgcgtgctggcaacaaatgataggggttgcgctcgttgcgggacttaacccaacatctcacgacacgagctgacgacagccgtgcaccacctgttttctcgtctcccgaaggagaacttctcatttctgaaaatagcgatcaatgtcaagacctggtaaggttcttcgcgttgcgtcgaattaaaccacatactccaccgcttgtgcgggcccccgtcaattcctttgagttttaatcttgcgatcgtacttcccaggcgggatacttattgcgttaactccggcacagaaggggtcgatacctcctacacctagtatccatcgtttacggcgtggactaccagggtatctaatcctgtttgctccccacgctttcgcgcctaagcgtcagttgtcgtccagaaagtcgccttcgccaccggtgttcttcctaatctctacgcatttcaccgctacactaggaattccactttcctctccgacactccagcttctcagtttccatcccatcacggggttaagccccgcacttttaagatggacttaagtagccgcctgcgcgcgctttacgcccaataattccggacaacgcttgccacctacgtattaccgcggctgctggcacgtagttagccgtggcttgctttttcggtaccgtcacttatgcagattattcgtctacttatctttcgtcccgaatcacagagttttacaacccgaaggccgtcttcactcacgcggcgttgctccgtcaggctttcgcccattgcggaagattccccactgctgcctcccgtaggagtctggaccgtgtctcagttccaatgtggccgttcatcctctcagaccggctaccgatcgtcggcttggtgggccgttacctcaccaactacctaatcggacgcaaacccctcttcaggcgatagcttacaagtagaggccaccctttctttcttgtgtcatgcgactcaagaatcgtattcggtattagcagtcgtttccgtctgttgtccccatcctgaaggcaggttgtttacgtgttactcacccgtttgccactggatttcataaagagcaagctcttcatgtcttcccgttcgacttgcatgtgttaagcacgccgccagcgttcgtcctgagccaggatcaaactctccatg

SEQ ID NO: 7 - DNA, 1563 bp, synthetic construct
  misc_feature 1..1563: note = Dialister
  source 1..1563: mol_type = other DNA
aggaggtgatccagccgcaccttccgatacggctaccttgttacgacttcactccaatcatcagctccgccttcgacggctggctccttacggttacctcaccggcttcgggtgtttctgactctcatagtgtgacgggcggtgtgtacaaggcccgggaacgtattcaccgcggtatgctgacccgcgattactagcgattcctgcttcatgcaggcgggttgcagcctgcaatctgaactgggggtgtgtttttgaggtttgctccgggtcgccccattgcttctctttgttgacacccattgtagtacgtgtgtagcccaggtcataaggggcatgatgacttgacgtcatccccgccttcctccgcgttgtccgcggcggtctctcatgagttcccaccattacgtgctggcaacataagataggggttgcgctcgttgcgggacttaacccaacatctcacgacacgagctgacgacagccgtgcaccacctgttttcctgtccccgaagggaactcccgatttctcgggattgcagtcaatgtcaagacctggtaaggttcttcgcgttgcgtcgaattaaaccacatactccaccgcttgtgcgggcccccgtcaattcctttgagttttaatcttgcgatcgtacttcccaggcggaatacttattgtgttagctccggcacagaaggggtcgatacctcctacacctagtattcatcgtttacggcgtggactaccagggtatctaatcctgtttgctccccacgctttcgcgcctcagcgtcagttgtcgtccagaaagtcgccttcgccaccggtgttcttcctaatctctacgcatttcaccgctacactaggaattccactttcctctccgatactccagcctcccagtttccatcccatcacggggttaagccccgcacttttaagatggacttaagaagccgcctgcgcgcgctttacgcccaataattccggacaacgcttgccacttacgtattaccgcggctgctggcacgtagttagccgtggcttgctattcgggtaccgtcactctgatatggtattagcataccagccattcgtccccgatcacagagctttacaacccgaaggccgtcatcactcacgcggcgttgctccgtcagactttcgtccattgcggaagattccccactgctgcctcccgtaggagtctggaccgtgtctcagttccagtgtggccgttcatcctctcagaccggctactgatcgtaggtttggtgggccgttacctcaccaactgcctaatcagacgcaaacccctcttcaggcgatagcttataaatagaggccatcctttcttccggaaatcatgcggtctccggaacacattcggtattagcagtcgtttccgtctgttgtccccatcctgaaggcaggttgtttacgtgttactcacccgtttgccactagattcaaaaagaagcaagcttctcttttcctcccgttcgacttgcatgtgttaagcacgccgccagcgttcgtcctgagccaggatcaaactctccatg

SEQ ID NO: 8 - DNA, 1110 bp, synthetic construct
  misc_feature 1..1110: note = Phascolarctobacterium
  source 1..1110: mol_type = other DNA
tggagagtttgatcctggctcaggacgaacgctggcggcatgcctaacacatgcaagtcgaacggagattatttcggtaatcttagtggcgaacgggtgagtaacgcgtaggcaacctgccctttagttggggacaacatcccgaaaggggtgctaataccgaatgtgacagtttttccgcatgggattactgtgaaagatggcctctatttataagctatcgctaaaggatgggcctgcgtctgattagctagttggtggggtaacggcctaccaaggcgacgatcagtagccggtctgagaggatgaacggccacattgggactgagacacggcccagactcctacgggaggcagcagtggggaatcttccgcaatgggcgaaagcctgacggagcaatgccgcgtgagtgatgaaggaattcgttccgtaaagctcttttgtttatgacgaatgtgcagactgtgaataatggtttgtaatgacggtagtaaacgaataagccacggctaactacgtgccagcagccgcggtaatacgtaggtggcgagcgttgtccggaattattgggcgtaaagagcatgtaggcggttttttaagtccggagtgaaaatgcggggctcaaccccgtatggctctggatactggaagacttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacgtccgcaaggatgaaactcaaaggaattgacgggggcccgcacaagcggtggagtatgtggtttaattcgacgcaacgcgaagaaccttaccaaggcttgacattgagtgaccgctctagagatagagcttcttcttcggaagcacgaaaacaggtggtgcatggctgtcgtcagctcgtgtcgtgagatgttgggttaagtcccgcaacgag

SEQ ID NO: 9 - DNA, 1655 bp, synthetic construct
  misc_feature 1..1655: note = Phascolarctobacterium
  source 1..1655: mol_type = other DNA
aggaggtgatccagccgcaccttccgatacggctaccttgttacgacttcaccccaatcatcggccccaccttagacggctggctccttacggttaccccaccggcttcgggtgttaccaactttcgtggtgtgacgggcggtgtgtacaaggcccgggaacgtattcaccgcagtatgctgacctgcgattactagcgattccgacttcatgcaggcgagttgcagcctgcaatccgaactgggatcgggtttctggggtttgctctgcctcacggctttgcttccctctgttgccgaccattgtagtacgtgtgtagcccaagacataaggggcatgatgacttgacgtcatccccgccttcctccaagttgtccttggcagtctcctatgagtccccggcattacccgctggtaacataggataggggttgcgctcgttgcgggacttaacccaacatctcacgacacgagctgacgacagccatgcaccacctgttttcatgtccccgaagggaaagctccatctctggagcggtcattcaatgtcaagccttggtaaggttcttcgcgttgcgtcgaattaaaccacatactccaccgcttgtgcgggcccccgtcaattcctttgagtttcatccttgcggacgtactccccaggcggggtacttattgcgttaactccggcacagaaggggtcgatacctcctacacctagtacccatcgtttacggccaggactaccggggtatctaatcccgttcgctaccctggctttcgcatctcagcgtcagacacagtccagaaaggcgccttcgccactggtgttcctcccaatatctacgcatttcaccgctacactgggaattcccctttcctctcctgcactcaagtcttccagtatccagagccatacggggttgagccccgcattttcactccagacttaaaaaaccgcctacatgctctttacgcccaataattccggacaacgctcgccacctacgtattaccgcggctgctggcacgtagttagccgtggcttattcgtttactaccgtcattacagatcattattcacaatctgcacattcgtcataaacaaaagagctttacggaacgaattccttcatcactcacgcggcattgctccgtcaggctttcgcccattgcggaagattccccactgctgcctcccgtaggagtctgggccgtgtctcagtcccaatgtggccgttcatcctctcagaccggctactgatcatcgccttggtaggccgttaccccaccaactagctaatcagacgcaggcccatcctctagcgatagcttataaatagaggccatctttcacagcatcctcatgcgagattgctgtcacattcggtattagcacccctttcgggatgttgtccccatctagagggcaggttgcccacgcgttactcacccgttcgccactaagaaagttcaacacctaatacttcaaccaccgtgttagttcatgcttttaatgcgtagtaaaaattgcttcgcaatttttaccgctacacttcagcggatgaaatacttggtgttgaactttctccgttcgacttgcatgtgttaggcatgccgccagcgttcgtcctgagccaggatcaaactctccaata

SEQ ID NO: 10 - DNA, 1556 bp, synthetic construct
  misc_feature 1..1556: note = Phascolarctobacterium
  source 1..1556: mol_type = other DNA
tggagagtttgatcctggctcaggacgaacgctggcggcgtgcctaacacatgcaagtcgaacggagaattttatttcggtagaattcttagtggcgaacgggtgagtaacgcgtaggcaacctaccctttagacggggacaacattccgaaaggagtgctaataccggatgtgatcatcgtgccgcatggcaggatgaagaaagatggcctctacaagtaagctatcgctaaaggatgggcctgcgtctgattagctagttggtagtgtaacggactaccaaggcgatgatcagtagccggtctgagaggatgaacggccacattgggactgagacacggcccaaactcctacgggaggcagcagtggggaatcttccgcaatggacgaaagtctgacggagcaacgccgcgtgagtgatgaaggatttcggtctgtaaagctctgttgtttatgacgaacgtgcagtgtgtgaacaatgcattgcaatgacggtagtaaacgaggaagccacggctaactacgtgccagcagccgcggtaatacgtaggtggcgagcgttgtccggaattattgggcgtaaagagcatgtaggcggcttaataagtcgagcgtgaaaatgcggggctcaaccccgtatggcgctggaaactgttaggcttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacggccgcaaggttgaaactcaaaggaattgacgggggcccgcacaagcggtggagtatgtggtttaattcgacgcaacgcgaagaaccttaccaaggcttgacattgattgaacgctctagagatagagatttcccttcggggacaagaaaacaggtggtgcatggctgtcgtcagctcgtgtcgtgagatgttgggttaagtcccgcaacgagcgcaacccctatcctatgttaccagcaagtaaagttggggactcatgggagactgccagggacaacctggaggaaggcggggatgacgtcaagtcatcatgccccttatgtcttgggctacacacgtactacaatggtcggaaacagagggaagcgaagccgcgaggcagagcaaaccccagaaacccgatctcagttcggatcgcaggctgcaacccgcctgcgtgaagtcggaatcgctagtaatcgcaggtcagcatactgcggtgaatacgttcccgggccttgtacacaccgcccgtcacaccacgaaagttggtaacacccgaagccggtgaggtaacctattaggagccagccgtctaaggtggggccgatgattggggtgaagtcgtaacaaggtagccgtatcggaaggtgcggctggatcacctcctttc

SEQ ID NO: 11 - DNA, 22 bp, synthetic construct
  misc_feature 1..22: note = Dialister primer 1
  source 1..22: mol_type = other DNA
gagtatcggagaggaaagtgga

SEQ ID NO: 12 - DNA, 23 bp, synthetic construct
  misc_feature 1..23: note = Dialister primer 2
  source 1..23: mol_type = other DNA
gcttctctttgttgacacccatt

SEQ ID NO: 13 - DNA, 22 bp, synthetic construct
  misc_feature 1..22: note = Phascolarctobacterium primer 1
  source 1..22: mol_type = other DNA
ggagtgctaataccggatgtga

SEQ ID NO: 14 - DNA, 20 bp, synthetic construct
  misc_feature 1..20: note = Phascolarctobacterium primer 2
  source 1..20: mol_type = other DNA
ccgtggcttcctcgtttact

SEQ ID NO: 15 - DNA, 17 bp, synthetic construct
  misc_feature 1..17: note = primer 341F
  misc_difference 9: note = note = n is a, c, g, or t
  source 1..17: mol_type = other DNA
cctacgggnbgcascag

SEQ ID NO: 16 - DNA, 20 bp, synthetic construct
  misc_feature 1..20: note = primer 806bR
  misc_difference 8: note = n is a, c, g, or t
  source 1..20: mol_type = other DNA
ggactacnvgggtwtctaat

SEQ ID NO: 17 - DNA, 20 bp, synthetic construct
  source 1..20: mol_type = other DNA
cacrgtaaacgatggatgcc

SEQ ID NO: 18 - DNA, 16 bp, synthetic construct
  source 1..16: mol_type = other DNA
ccagacgttgggctgg